(19) United States
(12) Patent Application Publication
(10) Pub. No.: US 2024/0404675 A1
(43) Pub. Date: Dec. 5, 2024

(54) Artificial Intelligence System Using Rule-Based Preprocessing

(71) Applicant: CVS Pharmacy, Inc., Woonsocket, RI (US)

(72) Inventors: Romain Bogaerts, Providence, RI (US); Neil Foglio, Cranston, RI (US); Avinash Gopinath, Wellesley, MA (US); Taras Gorishnyy, Woonsocket, RI (US); Thao Ngo, Millbury, MA (US); Ray M. Sehgal, Milton, MA (US); Simon Zahn, Boston, MA (US); David Ramos, Seekonk, MA (US); Gulzar Virk, Libertyville, IL (US); Joshua Cambria, Rehoboth, MA (US); Anirban Basu, Mansfield, MA (US)

(21) Appl. No.: 18/486,313

(22) Filed: Oct. 13, 2023

Related U.S. Application Data
(63) Continuation-in-part of application No. 17/573,435, filed on Jan. 11, 2022.
(60) Provisional application No. 63/416,300, filed on Oct. 14, 2022.

(51) Int. Cl. G16H 20/13; G06Q 10/00; G16H 40/20

(52) U.S. Cl. CPC G16H 20/13; G06Q 10/00; G16H 40/20

(57) ABSTRACT

Systems and methods for using rule-based preprocessing to improve artificial intelligence prediction of next best actions are disclosed. Prescription information may be used to identify a parameter set for a machine learning model. The parameter set may first be processed through a rules engine having a first rule set configured to conditionally determine the next best action service call without processing through the machine learning model. The machine learning model may then selectively process the parameter set to determine the next best action service call if the first rule set did not meet a next best action condition in the rule set. The next best action service call may be initiated to execute the next best action. For example, in response to rejection from insurance adjudication, the next best action may change the prescription information for automatically resubmitting the prescription to insurance adjudication.

### FIELD OF THE DISCLOSURE

The present disclosure relates to systems and methods for using artificial intelligence systems to optimize the fulfillment of prescriptions. In particular, the present disclosure relates to using an artificial intelligence system with rules-based preprocessing to improve machine learning model processing.

### BACKGROUND

Presently there are many sources of conflict, delay, and other issues in the prescription fulfillment workflow. Conventional resolutions have tended to be linear in resolution with the issues typically being identified when one step of the linear workflow is unable to be completed as anticipated. Current pharmacy workflows typically include a sequential process of steps that includes a sequence of evaluations with a subsequent evaluation performed only after any issues in the preceding step have been resolved. Such a sequential approach is time-consuming and inefficient when issues arise in the fulfillment of a prescription.

For example, one step in the sequential process is a stock check. A stock check process is implemented to determine if there is a sufficient inventory to fill the prescription. When a problem or exception is identified due to the medication being out of stock, an out-of-stock resolution process orders additional stock for the medication without regard for or knowledge of other problems that may be encountered when trying to fulfill the particular prescription. Such a conventional exception process then may result in a delay in the therapy due to an unforeseen backorder, as well as other additional sequential delays because of the other problems in fulfilling the prescription, leading to much longer delay.

Another example of a step in the sequential process is an adjudication of the prescription. An adjudication process submits a prescription claim to a third-party payor's insurance adjudication system for approval and may result in a subsequent problem or exception because the patient lacks any third-party insurance coverage to pay for the medication. In an attempt to solve the subsequent exception, a pharmacist may seek to modify the prescription, cash out the claim, or request a therapeutic alternate to the prescribed medication from the prescribing doctor. Resolution of this subsequent exception is solely at the pharmacist's discretion, subject to the pharmacist's availability, and knowledge of less expensive alternatives. Additionally, if none are available, the prior steps in the process were performed unnecessarily.

Considering the above, there is currently no comprehensive solution for avoiding increased workload for handling ordered medication that may not be dispensed or providing an improved workflow for prescription management and fulfillment.

### SUMMARY

An artificial intelligence system using rule-based preprocessing to improve the automated processing efficiency of pharmacy computing systems that support prescription management and fulfilment is described. In some configurations, automated execution of pre-model rule sets and/or one or more special resolution models prior to execution of a general resolution model may reduce the computational processing resources allocated to processing each prescription and accelerate the automated resolution of errors, particularly as they relate to insurance coverage.

In one aspect, a computer-implemented method includes: receiving prescription information comprising a prescription for a patient; identifying, based on the prescription information, a parameter set for determining a next best action service call from a set of next best action services; processing the parameter set using a rules engine and a first rule set configured to conditionally determine the next best action service call in response to the parameter set meeting a next best action condition in the first rule set; selectively processing, using a first machine learning model trained for the set of next best action services, the parameter set to determine the next best action service call; and initiating the next best action service call.

Examples may include one or more of the following features. Identifying the parameter set may include determining insurance coverage parameters for the prescription and the patient. The computer-implemented method may include: selectively processing, using a second machine learning model trained for a first next best action service from the set of next best action services, the parameter set to determine a first confidence value for the first next best action service; and determining, based on the first confidence value, whether to determine the next best action service call to be for the first next best action service or selectively process the parameter set using the first machine learning model. The computer-implemented method may include: receiving, responsive to submitting the prescription information to insurance adjudication, a rejection notification may include a rejection code from a plurality of rejection codes; and adding the rejection code to the parameter set. The computer-implemented method may include: selectively processing, using a plurality of specialized machine learning models, the parameter set to determine confidence values corresponding to each specialized machine learning model of the plurality of specialized machine learning models, where each specialized machine learning model is trained for a target rejection code of the plurality of rejection codes; and generates a confidence value corresponding to a likelihood that a next best action service corresponding to that specialized machine learning model is the next best action service; and determining, based on the confidence values, whether to determine the next best action service call to be for a next best action service corresponding to one of the specialized machine learning models or selectively process the parameter set using the first machine learning model. Selectively processing the parameter set using the plurality of specialized machine learning models may be executed responsive to processing the parameter set using the first rule set and prior to processing the parameter set using the first machine learning model. The plurality of specialized machine learning models may include at least one specialized machine learning model configured to generate a change parameter for use in the next best action service call. The computer-implemented method may include processing the parameter set using the rules engine and a second rule set configured to validate the next best action service call determined by the first machine learning model prior to initiating the next best action service call. The set of next best action services may include at least two services selected from: therapeutic alternative recommendation service; a quantity change service; an insurance plan change service; a cash pay change service; a future fill date change service; a prescriber request service; a prior authorization request service; and a customer service prompt service. The computer-implemented method may include automatically submitting, responsive to completing the next best action service call, updated prescription information to insurance adjudication.

In another aspect, a system includes at least one processor and a memory. The memory stores instructions which, when executed, cause the at least one processor to: receive prescription information comprising a prescription for a patient; identify, based on the prescription information, a parameter set for determining a next best action service call from a set of next best action services; process the parameter set using a rules engine and a first rule set configured to conditionally determine the next best action service call in response to the parameter set meeting a next best action condition in the first rule set; selectively process, using a first machine learning model trained for the set of next best action services, the parameter set to determine the next best action service call; and initiate the next best action service call.

Examples may include one or more of the following features. Identifying the parameter set may include determining insurance coverage parameters for the prescription and the patient. The instructions may cause the at least one processor to: selectively process, using a second machine learning model trained for a first next best action service from the set of next best action services, the parameter set to determine a first confidence value for the first next best action service; and determine, based on the first confidence value, whether to determine the next best action service call to be for the first next best action service, or selectively process the parameter set using the first machine learning model. The instructions may cause the at least one processor to: receive, responsive to submitting the prescription information to insurance adjudication, a rejection notification may include a rejection code from a plurality of rejection codes; and add the rejection code to the parameter set. The instructions may cause the at least one processor to: selectively process, using a plurality of specialized machine learning models, the parameter set to determine confidence values corresponding to each specialized machine learning model of the plurality of specialized machine learning models, where each specialized machine learning model is trained for a target rejection code of the plurality of rejection codes and generates a confidence value corresponding to likelihood that a next best action service corresponding to that specialized machine learning model is the next best action service; and determine, based on the confidence values, whether to determine the next best action service call to be for a next best action service corresponding to one of the specialized machine learning models or selectively process the parameter set using the first machine learning model. At least one processor may be configured to execute the instructions to selectively process the parameter set using the plurality of specialized machine learning models: responsive to processing the parameter set using the first rule set; and prior to processing the parameter set using the first machine learning model. The plurality of specialized machine learning models may include at least one specialized machine learning model configured to generate a change parameter for use in the next best action service call. The instructions may cause the at least one processor to process the parameter set using the rules engine and a second rule set configured to validate the next best action service call determined by the first machine learning model prior to initiating the next best action service call. The instructions may cause the at least one processor to automatically submit, responsive to completing the next best action service call, updated prescription information to insurance adjudication.

In still another aspect, a system may include at least one processor and a memory. The memory stores instructions which, when executed, cause the at least one processor to: determine a set of next best action services; determine a training data set comprising of parameter sets for a plurality of prescriptions for a plurality of patients; label the training data set for the set of next best action services; iteratively train a first machine learning model to correlate parameter sets to predicting a next best action service from the set of next best action services; receive prescription information comprising a prescription for a patient; identify, based on the prescription information, a target parameter set for determining a next best action service call from the set of next best action services; process the parameter set using a rules engine and a first rule set configured to conditionally determine the next best action service call in response to the parameter set meeting a next best action condition in the first rule set; selectively process, using the first machine learning model, the parameter set to determine the next best action service call; and initiate the next best action service call.

### BRIEF DESCRIPTION OF THE DRAWINGS

The techniques introduced herein are illustrated by way of example, and not by way of limitation in the figures of the accompanying drawings in which like reference numerals are used to refer to similar elements.

FIG. 1 is a block diagram illustrating a pharmacy system including a prescription optimization system in accordance with some implementations.

FIG. 2 is a block diagram illustrating the prescription optimization system in accordance with some implementations.

FIG. 3 is a diagram showing the data flow through the prescription optimization system in accordance with some implementations.

FIG. 4 is a block diagram illustrating a problem identification module in accordance with some implementations.

FIG. 5 is a block diagram illustrating a therapeutic alternative recommendation module in accordance with some implementations.

FIG. 6 illustrates an example therapeutic alternative list of the alternative drug data store in accordance with some implementations.

FIG. 7 is a block diagram illustrating an example of a next best action module in accordance with some implementations.

FIG. 8 is a flow diagram showing a method for optimizing fulfillment of a prescription in accordance with some implementations.

FIG. 9 is a flow diagram showing a method for identifying a fulfillment problem in accordance with some implementations.

FIG. 10 is a flow diagram showing a method for determining a therapeutic alternative in accordance with some implementations.

FIG. 11 is a flow diagram showing a method for determining a next best action in accordance with some implementations.

FIGS. 12 and 13 are graphic representations of example user interfaces for a pharmacist to interact with the pharmacy system in accordance with some implementations.

FIGS. 14-17 are graphic representations of example user interfaces for a patient to interact with the pharmacy system in accordance with some implementations.

FIG. 18 is a block diagram illustrating an example of an insurance coverage module in accordance with some implementations.

FIG. 19 is a block diagram illustrating an example of a machine learning training module in accordance with some implementations.

FIG. 20 is a flow diagram showing a method for optimizing fulfillment of a prescription in accordance with some implementations.

FIG. 21 is a flow diagram showing a method for determining a next best action following a rejection from an insurance adjudication system in accordance with some implementations.

FIG. 22 is a flow diagram showing a method for training machine learning models for determining next best actions in accordance with some implementations.

### DETAILED DESCRIPTION

As set forth in detail below, the technology described herein provides an innovative approach to evaluate a prescription and provide proactive processing in a workflow to reduce conflicts in dispensing medication to a patient or customer. In particular, the systems and methods described below advantageously optimize problem resolution to ensure that unnecessary steps are eliminated from the process of fulfilling prescriptions. In some implementations, the systems and methods determine and perform the next best action to fulfill a prescription. In some implementations, the systems and methods described herein are configured to evaluate a prescription for treatment of a patient or customer and to identify a list of therapeutic alternatives to the prescription. Based on identifying these alternatives, resources may be reduced, and the practicalities of therapeutic alternatives may be identified in advance of the patient acquiring the prescribed medication.

In some implementations, an insurance coverage module may implement a third-party rejection resolution (TPRR) algorithm to reduce the delay that may be caused by resolvable errors in the prescription information. This TPRR algorithm may be implemented in an artificial intelligence system configured to automatically resolve and resubmit payment requests to insurance adjudication systems maintained by third party payors for determining whether a given prescription is covered under their insurance plan for that patient. The TPRR algorithm may be part of a set of pharmacy support tools for optimizing problem resolution in the process of fulfilling prescriptions and may use machine learning models to automate the adjudication process, particularly the automated handling of next best actions following a rejection notification from the third-party payor, with a goal of automated resubmission and approval. Rejection from adjudication may result in both pharmacy and patient disruption and the manual handling by pharmacy staff may result in inconsistent and suboptimal resolution. Automation of the adjudication process may reduce manual work, optimize patient outcomes, and enhance the filling experience.

However, using machine learning models to automate a complex decision-making process may benefit from a general resolution machine learning model being supported by a logical rules engine (based on Boolean logic, rather than machine learning algorithms) configured with one or more rule sets to reduce the edge cases with known next best actions and/or validate that the next best action determinations made by machine learning models comply with logical, regulatory, and/or third-party provider conditions. For example, a rules engine may pre-filter for certain rejection codes and/or specific next best actions that have either a direct logical outcome based on the rejection code and one or more prescription parameter values and/or require manual intervention. The rules engine may also run rules following determination of a next best action by the machine learning models that can override the models' outputs and generate alternative next best actions. The pre and/or post-processing by the rules engine may reduce the set of next best actions that the general resolution machine learning model needs to be trained to and support production processing of. In some implementations, machine learning models for each next best action may be integrated and tested with pre-model and post-model rule sets to increase the efficiency and accuracy of the automated selection of next best action service calls.

Further, performance of the general resolution machine learning model may be improved by using one or more special resolution machine learning models trained to evaluate specific rejection codes and/or next best actions and further reduce the conditions under which the general resolution machine learning model is used. In some implementations, specialized machine learning models may also be integrated that generate parameter changes for use in implementing the next best action. These specialized resolution models, including parameter change models, may improve both processing efficiency of the combined models in the artificial intelligence system and the accuracy of the next best action predictions.

Some aspects described herein provide improved compliance with prescribed treatments for a patient. For example, a prescription intake module 142 is configured to process a prescription for treatment of the patient. The problem identification module 144 is configured to identify the problems that may be encountered in filling the prescription. The therapeutic alternative recommendation module 146 is configured to identify a list of therapeutic alternatives to the prescription for the treatment of the patient. A next best action module 150 is configured to generate a respective next best action for the prescription and the therapeutic alternatives for the patient in the ranked list. An action execution module 152 is coupled to receive the next best action from the next best action module 150 and perform it.

This approach allows a physician and/or pharmacist to provide the patient with clinically appropriate and cost-effective prescribed treatment options, and fulfill the prescription in an efficient and time effective manner. These and other features may provide a substantially better experience for patients, healthcare providers, and insurance providers. In particular, the techniques introduced herein may drive improved utilization of the best insurance covered drugs for the patient and plan. The benefits of the techniques include, for example, maximizing the use of a formulary benefit and decreased expenses on medications for the patient, improved formulary compliance, reduced spend on covered medications for the payor, and increased ability to drive adherence (e.g., minimizing delays in first fill and/or refills, minimizing cost barriers, etc.) for the pharmacy.

With reference to the figures, reference numbers may be used to refer to components found in any of the figures, regardless whether those reference numbers are shown in the figure being described. Further, where a reference number includes a letter or number, generally separated by a decimal point, referring to one of multiple similar components (e.g., component 000.a, 000.1, and 000.n), the reference number may be used without the number or letter after the decimal point to refer to one or all of the similar components.

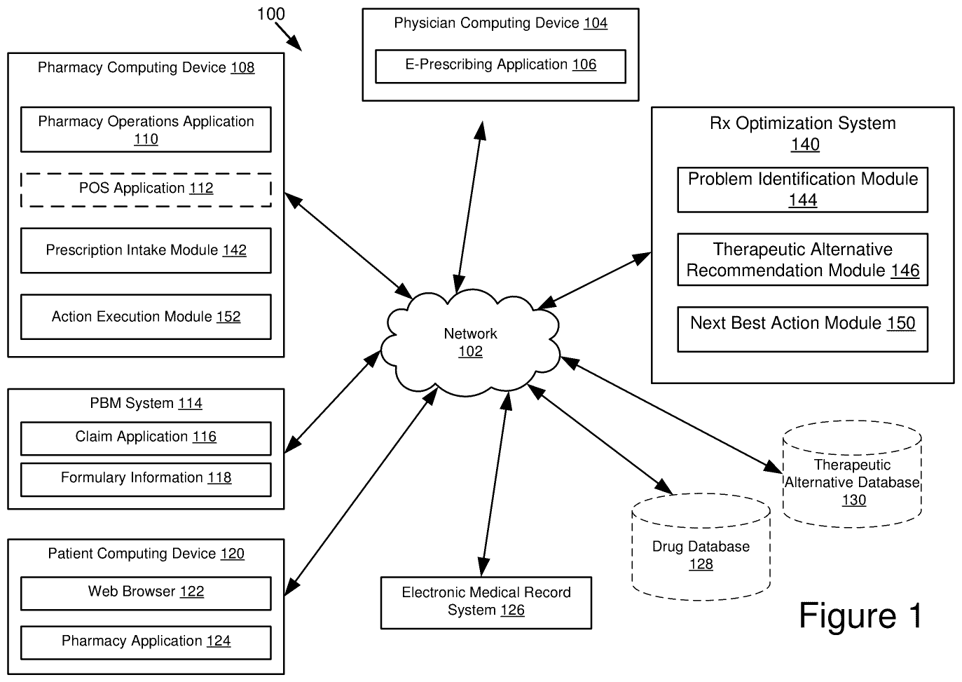

FIG. 1 is a high-level block diagram illustrating an example pharmacy system 100 for fulfilling a prescription. The pharmacy system 100 includes a network 102, a physician computing device 104, a pharmacy computing device 108, a pharmacy benefit management (PBM) system 114, a patient computing device 120, an electronic medical record system 126, a drug database 128, a therapeutic alternative database 130, and a prescription (Rx) optimization system 140. While a particular arrangement is depicted in FIG. 1 by way of example, it should be noted that other system configurations are possible including other devices, systems, and networks as well as pluralities of any of the components shown in FIG. 1.

The network 102 may communicatively couple the various components of the system 100. In some implementations, the network 102 is a wired or wireless, and may have numerous different configurations. Furthermore, the network 102 may include a local area network (LAN), a wide area network (WAN) (e.g., the internet), and/or other interconnected data paths across which multiple devices may communicate. In some implementations, the network 102 may be a peer-to-peer network. The network 102 may also be coupled with portions of a telecommunications network for sending data using a variety of different communication protocols. In some implementations, the network 102 may include Bluetooth (or Bluetooth low energy) communication networks or a cellular communications network for sending and receiving data including via short messaging service (SMS), multimedia messaging service (MMS), hypertext transfer protocol (HTTP), direct data connection, wireless access point (WAP), email, etc. Although the example of FIG. 1 illustrates one network 102, in practice one or more networks can connect the entities of the system 100.

The physician computing device 104 may include one or more computing devices having data processing and communication capabilities. The physician computing device 104 is coupled to communicate with other components of the system 100 via network 102. While the example of FIG. 1 depicts only a single physician computing device 104, the system 100 may include any number of physician computing devices 104. The physician computing device 104 may include an e-prescribing application 106. An e-prescribing application 106 provides functionality for a physician using the physician computing device 104 to enter a prescription and treatment information for a patient. The e-prescribing application 106 allows the prescription to be electronically stored and/or communicated (e.g., transmitted to a pharmacy, stored to an electronic medical record for the patient, etc.). In some implementations, the e-prescribing application 106 may be part of a broader practice management software suite used by a physician.

The pharmacy computing device 108 may include one or more computing devices having data processing and communication capabilities. The pharmacy computing device 108 is coupled to communicate with other components of the system 100 via network 102. While the example of FIG. 1 depicts only a single pharmacy computing device 108, the pharmacy system 100 may include any number of pharmacy computing devices 108. In some implementations, the pharmacy computing device 108 includes a pharmacy operations application 110, a point-of-sale (POS) application 112, a prescription intake module 142, an action execution module 152. The pharmacy operations application 110 is used by the pharmacist or pharmacy technician to manage the prescription fulfillment process. For example, the pharmacy operations application 110 receives and processes changes and edits made by the pharmacist such as quantity, error in prescription, typos, etc. The pharmacy operations application 110 also triggers the prescription being sent to the prescription optimization system 140 once the prescription intake module 142 has processed the prescription, such as retrieving adjudication information or sending the prescription to the PBM system 114 to perform adjudication and receiving the results. In some implementations, the pharmacy computing device 108 may be used as a POS system (not shown) or include an optional POS application 112. The POS application 112 facilities processing payments from the patients for their prescription or their insurance copays. The prescription intake module 142 receives a prescription for a patient. The prescription intake module 142 is coupled to other devices or systems 104, 114, 120 to receive prescription information or prescriptions for patients. The prescription intake module 142 may be steps, processes, functionalities, software executable by a processor or a device including routines for receiving prescription, retrieving information related to it, and sending it to other components of the prescription optimization system 140 for further processing. For example, the physician computing device 104 sends electronic prescriptions or issues prescriptions via fax and they are received by the prescription intake module 142 so the physician can write a prescription by hand and then that prescription gets sent to prescription optimization system 140 by the prescription intake module 142. The prescription intake module 142 can process written scripts, phone scripts, fax scripts, or electronic scripts. In some implementations, the prescription intake module 142 ensures that the prescription is digitized and input into a processing queue of the pharmacy operations application 110. In some implementations, the prescription intake module 142 also processes the received prescription and interfaces with systems such as the PBM system 114 to perform claim adjudication on the prescription. For example, the prescription intake module 142 may retrieve coverage status and cost of the medicine parameters (e.g., co-pay amount) for the prescription. The pharmacy computing device 108 may also include an action execution module 152. The action execution module 152 is coupled to receive a next best action from the prescription optimization system 140 and cooperate with the pharmacy operations application 110 to execute the action specified. The actions to be executed or performed are described in more detail below with the description of the prescription optimization system 140.

The pharmacy benefit management (PBM) system 114 may include one or more computing devices having data processing and communication capabilities. The pharmacy benefit management system 114 may be coupled to communicate with other systems, devices and databases of the system 100 via network 102. The pharmacy benefit management system 114 may also be coupled to an insurance provider system (not shown) for submitting and processing claims and determining insurance adjudication. The pharmacy benefit management system 114 may include a claim application 116 and formulary information 118. The claim application 116 may be configured to process requests from a pharmacy operations application 110, prescription intake module 142, and/or and e-prescribing application 106. The claim application 116 can process the requests, provide them to an insurance adjudication system of an insurance company or benefit provider and return information of coverage, denial and other insurance related information, which may include rejection codes for denials. In some configurations, problem identification model 144 may respond to a claim rejection and next best action module 150 may determine one or more actions before initiating a resubmission service call to claim application 116. The formulary information 118 includes a list of prescription medications that are approved to be prescribed to patients covered by a particular insurance policy or insurer. The formulary information 118 may include formularies for any number of insurance policies as will be described in further detail below.

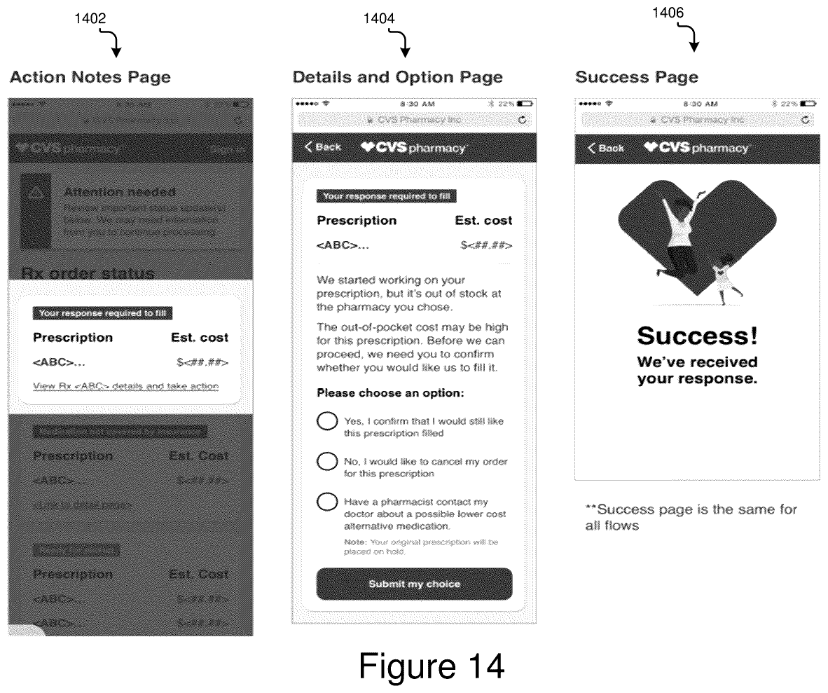

The patient computing device 120 may include one or more computing devices having data processing and communication capabilities. The patient computing device 120 may be coupled to communicate with other systems, devices and databases of the system 100 via network 102. While the example of FIG. 1 depicts only a single patient computing device 120, the system 100 may include any number of patient computing devices 120. The patient computing device 120 may include a web browser 122 and/or a pharmacy application 124. The web browser 122 and/or pharmacy application 124 provide functionality for a patient using the patient computing device 120 to look up a treatment that is being prescribed or has been prescribed to the patient to determine whether the treatment is cost effective or whether there may be more cost-effective treatments to discuss with a prescriber and/or pharmacist. According to the techniques introduced herein, the web browser 122 may connect to the prescription optimization system 140 and provide an interface for the patient to view a list of clinically appropriate therapeutic alternatives for a prescribed treatment. Example user interfaces will be described below with reference to FIGS. 14-17. In some implementations, a dedicated pharmacy application 124 on the patient computing device 120 may provide the interface for the patient to view the list of clinically appropriate therapeutic alternatives for a prescribed treatment. The list may provide information for the patient to check for lower cost treatment options, or to confirm that their current treatment is the lowest cost option, while talking with their prescriber or pharmacist about their treatment plan. In some implementations, the patient computing device 120 is configured to communicate with the prescription optimization system 140 to show the patient problems that have occurred during fulfillment of a prescription, status of fulfillment, solicit information to address problems and provide input from the user to the prescription optimization system 140 to address the problem. The operation and interaction of the patient computing device 120 with the prescription optimization system 140 is described in more detail below.

The electronic medical record system 126 includes one or more computing devices having data processing and communication capabilities. The electronic medical record system 126 may be coupled to communicate with other systems, devices and databases of the system 100 via network 102. The electronic medical record system 126 also includes an associated memory for storing data records and computer program instructions. The electronic medical record system 126 may be configured to accurately store data relating to a patient and record the state of the patient over time. For example, electronic medical record system 126 may include records for patients that include information typically found in a paper chart, such as medical history, diagnoses, medications, immunization dates, allergies, and the like. Although only one electronic medical record system 126 is shown in FIG. 1, it should be understood that the pharmacy system may be coupled for communication with any number of electronic medical record systems 126 even for a single patient. For example, a patient may have records at a plurality of different electronic medical record systems 126; one for general care, one for a hospital, one for a medical specialist, one for a clinic, etc.

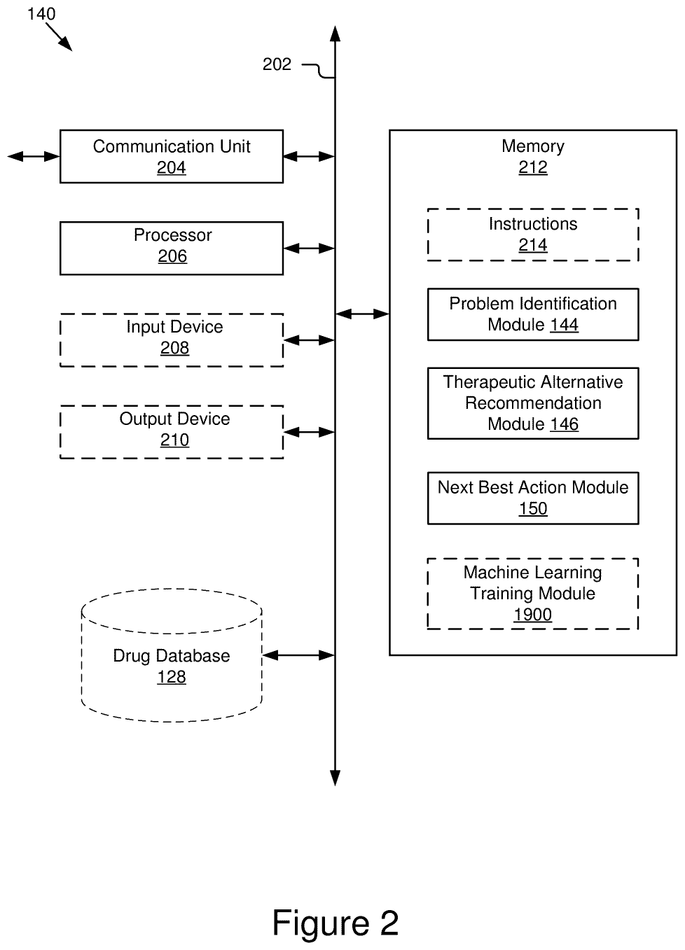

The drug database 128 may include one or more data stores that include information about prescription drugs and over-the-counter medications. In particular, the drug database 128 may include proprietary or in-house databases maintained by pharmacies or drug manufacturers, commercially available databases, and/or databases operated by a government agency. The drug database 128 may be accessed by using industry standard drug identifiers, such as and without limitation, a generic product identifier (GPI), generic sequence number (GSN), national drug code directory (NDC), universal product code (UPC), health related item, or manufacturer. In some implementations, the drug database 128 may be coupled to the systems, devices and databases of the pharmacy system 100 via the network 102. In some implementations, the drug database 128 may be partitioned and be separate from the data used for normal pharmacy operations by the pharmacy computing device 108. In such an implementation, the drug database 128 is part of the prescription optimization system 140 as shown in FIG. 2. Because of these alternate implementations, the drug database 128 is shown with dashed lines in FIGS. 1 and 2. In some implementations (not shown), the drug database 128 may be part of the pharmacy computing device 108. The drug database 128 can include one or more non-transitory computer-readable media for storing the data. In some implementations, the drug database 128 may be incorporated with the memory 212 or may be distinct therefrom. In some implementations, the drug database 128 may include a database management system (DBMS). For example, the DBMS could include a structured query language (SQL) DBMS, a NoSQL DBMS, various combinations thereof, etc. In some implementations, the DBMS may store data in multi-dimensional tables comprised of rows and columns, and manipulate, e.g., insert, query, update and/or delete, rows of data using programmatic operations.

In some implementations, the drug database 128 may also include other pharmacy information. The drug database 128 may store historical data from the pharmacy computing device 108 or other systems. As noted above, the information in the drug database 128 may be copied from the pharmacy computing device 108 and maintained separately so that a larger amount of historical data can be used by the prescription optimization system 140. The drug database 128 may include historical patient information, prescription history information, clinical information, prescriber outreach history, and drug information. For example, the historical patient information may include information on each patient or the patient's attributes (e.g., conditions or diseases); how many times a patient paid with cash over the past 30, 60 or 90 days; how many times a patient paid with their primary insurance, the connection of a payment in cash to the drug type, etc. As another example, the prescription history information may include a historical record of what prescriptions a particular patient had and when the patient had them, when the prescription was refilled (or not) as an indicium of adherence, etc. In yet another example, the clinical information may include source data that comes from the pharmacy computing device 108 (what happened in the actual pharmacy) but the data may be modified or optimized before storage in the drug database 128 so that it can be used by the prescription optimization system 140 and other systems and applications. For example, the prescriber outreach history may include any contact by the pharmacist or pharmacy technician to the prescribing physician and when outreach took place as well as whether there was patient outreach at or near the same time. Additionally, the prescriber outreach history may include whether there was any prescriber outreach different from patient outreach or outreach for refill authorization. Such independent prescriber outreach events can be used by the prescription optimization system 140 for better decision-making. For example, the drug information may include the specific drugs that are prescribed, the quantity of each drug as well as other drugs that are prescribed for particular patient, and whether generics or brand names were used to fill the prescription. This information can be used to develop specific insights for selecting and applying rules on specific decisions made by the prescription optimization system 140.

The therapeutic alternative database 130 may include one or more data stores that include information about prescription drugs, over-the-counter medications and alternatives to them. In some implementations, the therapeutic alternative database 130 is indexed based on the prescription for the treatment of the patient. For example, the therapeutic alternative database 130 may be indexed based on a national drug code (NDC) for the prescription for the treatment of the patient. Based on the NDC, other index or broader classification, the therapeutic alternative database 130 maintains a list of therapeutic alternatives mapped to the NDC. In some implementations, the therapeutic alternative database 130 may be customizable to include therapeutic alternatives determined based upon empirical observations. In some implementations, an entry for a therapeutic alternative is mapped and may include a doctor's instruction to the pharmacist on how the patient should use the medication (e.g., signature). In some implementations, an entry for the therapeutic alternative may be mapped to include a designation by the prescribing doctor to dispense the medication as written, or other information related to the prescription or drug. Similar to the drug database 128, the therapeutic alternative database 130 may be a database management system (DBMS). For example, the DBMS could include a structured query language (SQL) DBMS, a NoSQL DBMS, various combinations thereof, etc. In some implementations, the DBMS may store data in multi-dimensional tables comprised of rows and columns, and manipulate, e.g., insert, query, update and/or delete, rows of data using programmatic operations.

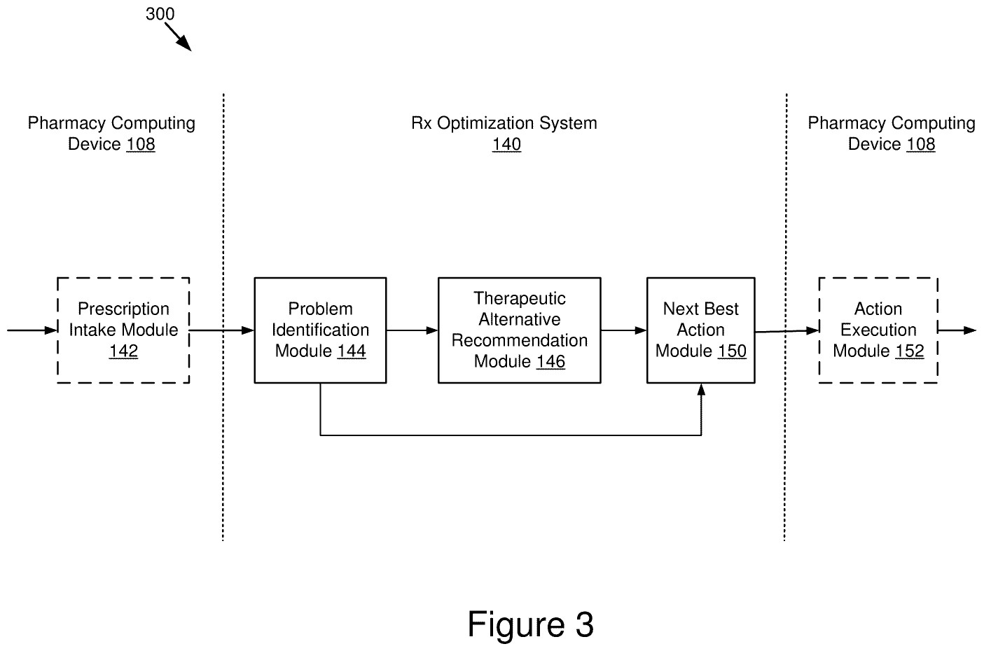

The prescription optimization system 140 includes one or more computing devices having data processing and communication capabilities as will be described in more detail below with reference to FIGS. 2-7 and 18-19. The prescription optimization system 140 may be coupled to communicate with other components of the system 100 via network 102. The prescription optimization system 140 comprises a problem identification module 144, a therapeutic alternative recommendation module 146, and a next best action module 150. The problem identification module 144 is configured to identify the problems that may be encountered in filling the prescription. The therapeutic alternative recommendation module 146 is configured to identify a list of therapeutic alternatives to the prescription for the treatment of the patient, to identify cost, availability, and/or exclusion, and rank the prescription and the list of the therapeutic alternatives. The next best action module 150 is configured to generate a respective next best action for the prescription and the therapeutic alternatives for the patient in the ranked list. Each of these components will be described in more detail below with reference to FIGS. 2 and 3.

Referring now to FIG. 2, one example for the prescription optimization system 140 is shown. In some implementations, the prescription optimization system 140 comprises a communication unit 204, a processor 206, an input device 208, an output device 210, and a memory 212, which may be communicatively coupled by a bus 202. The input device 208, the output device 210, the instructions 214, and machine learning training module 1900 are shown with dashed lines indicating they are optional. The prescription optimization system 140 depicted in FIG. 2 is provided by way of example and it should be understood that it may take other forms and include additional or fewer components without departing from the scope of the present disclosure. For instance, various components of the prescription optimization system 140 may be coupled for communication using a variety of communication protocols and/or technologies including, for instance, communication buses, software communication mechanisms, computer networks, etc. While not shown, the prescription optimization system 140 may include various operating systems, sensors, additional processors, and other physical configurations. The processor 206, memory 212, communication unit 204, etc., are representative of one or more of these components.

The bus 202 can include a communication bus for transferring data between components of the prescription optimization system 140, a network bus system including the network 102 or portions thereof, a processor mesh, a combination thereof, etc. In some implementations, the various components of the prescription optimization system 140 cooperate and communicate via a communication mechanism included in or implemented in association with the bus 202. In some implementations, the bus 202 may be a software communication mechanism including and/or facilitating, for example, inter-method communication, local function or procedure calls, remote procedure calls, an object broker (e.g., CORBA), direct socket communication (e.g., TCP/IP sockets) among software modules, UDP broadcasts and receipts, HTTP connections, etc. Further, communication between components of prescription optimization system 140 via bus 202 may be secure (e.g., SSH, HTTPS, etc.).

The communication unit 204 may include one or more interface devices (I/F) for wired and/or wireless connectivity among the components of the prescription optimization system 140 and the network 102. For instance, the communication unit 204 may include, but is not limited to, various types of known connectivity and interface options. The communication unit 204 may be coupled to the other components of the prescription optimization system 140 via the bus 202. The communication unit 204 can provide other connections to the network 102 and to other systems, devices and databases of the system 100 using various standard communication protocols.

The processor 206 may execute software instructions by performing various input, logical, and/or mathematical operations. The processor 206 may have various computing architectures to process data signals (e.g., CISC, RISC, etc.). The processor 206 may be physical and/or virtual, and may include a single core or plurality of processing units and/or cores. In some implementations, the processor 206 may be coupled to the memory 212 via the bus 202 to access data and instructions therefrom and store data therein. The bus 202 may couple the processor 206 to the other components of the prescription optimization system 140 including, for example, the communication unit 204, the input device 208, and the output device 210. The processor 206 is coupled by the communication unit 204 and the network 102 to retrieve and store information from the other components of the pharmacy system 100.

The input device 208 may include any device for inputting information into the prescription optimization system 140. In some implementations, the input device 208 may include one or more peripheral devices. For example, the input device 208 may include a keyboard, a pointing device, microphone, an image/video capture device (e.g., camera), a touch-screen display integrated with the output device 210, etc.

The output device 210 may be any device capable of outputting information from the prescription optimization system 140. The output device 210 may include one or more of a display (LCD, OLED, etc.), a printer, a 3D printer, a haptic device, audio reproduction device, touch-screen display, a remote computing device, etc. In some implementations, the output device 210 is a display which may display electronic images and data output by a processor, such as processor 206, of the prescription optimization system 140 for presentation to a user.

The memory 212 may store and provide access to data to the other components of the prescription optimization system 140. The memory 212 may be included in a single computing device or a plurality of computing devices. In some implementations, the memory 212 may store instructions 214 and/or data that may be executed by the processor 206. The memory 212 is also capable of storing other instructions and data, including, for example, an operating system, hardware drivers, other software applications, databases, etc. (not shown). The memory 212 may be coupled to the bus 202 for communication with the processor 206 and the other components of prescription optimization system 140. The memory 212 may include a non-transitory computer-usable (e.g., readable, writeable, etc.) medium, which can be any non-transitory apparatus or device that can contain, store, communicate, propagate or transport instructions 214, data, computer programs, software, code, routines, etc., for processing by or in connection with the processor 206. In some implementations, the memory 212 may include one or more of volatile memory and non-volatile memory (e.g., RAM, ROM, hard disk, optical disk, etc.). It should be understood that the memory 212 may be a single device or may include multiple types of devices and configurations.

In some implementations, the memory 212 also comprises the problem identification module 144, the therapeutic alternative recommendation module 146, the next best action module 150, and, optionally, the machine learning training module 1900. These modules, their configuration, structure and functionality are described below in more detail collectively and individually with reference to FIGS. 3-7 and 18-19. In some implementations, the problem identification module 144, the therapeutic alternative recommendation module 146, the next best action module 150, and/or machine learning training module 1900 are sets of instructions stored in the memory 212 executable by the processor 206 to provide their respective acts and/or functionality. In any of these implementations, the problem identification module 144, the therapeutic alternative recommendation module 146, or the next best action module 150 may be adapted for cooperation and communication with each other, the processor 206 and other components of the prescription optimization system 140 by the bus 202. The components 144, 146, and 150 are also coupled to the network 102 via the communication unit 204 for communication and interaction with the other systems, devices and databases of the pharmacy system 100. Although not shown in FIG. 2, in some alternate implementations the prescription intake module 142 and the action execution module 152 may be part of the prescription optimization system 140 and stored in memory 212.

The physician computing device 104, the pharmacy computing device 108, the pharmacy benefit management system 114, and the electronic medical record system 126 may be a computing device having a similar architecture as that shown in FIG. 2 but with their respective memories having the components noted in FIG. 1. In such implementations, the memory 212 may store one or more of the e-prescribing application 106, pharmacy operations application 110, POS application 112, claim application 116, formulary information 118, web browser 122, pharmacy application 124 and their respective components, depending on the configuration.

Referring now to FIG. 3, the flow 300 of data through the prescription optimization system 140 in accordance with some implementations is shown. The data flow shown begins with the prescription intake module 142 of the pharmacy computing device 108 receiving a prescription for a patient and processing it as has been described above with reference to FIG. 1. In some implementations, the prescription optimization system 140 may be part of the pharmacy computing device 108. The prescription intake module 142 then sends the prescription information including the prescription for the patient and related information to the problem identification module 144.

The problem identification module 144 may be steps, processes, functionalities, software executable by a processor, or a device including routines to identify problems that may be encountered in filling the prescription. The problem identification module 144 identifies one or more problems in fulfilling the prescription by analyzing the prescription information. In some implementations, the problem identification module 144 is particularly advantageous because it identifies and considers any problem that may occur in the fulfillment process through to completion. The problem identification module 144 is coupled to prescription intake module 142 to receive prescription information. The problem identification module 144 simultaneously identifies the potential problems that may arise during fulfillment of the prescription. The problem identification module 144 advantageously identifies any problems that may occur during fulfillment of the prescription thereby avoiding the problem of the prior art where problems are serially identified and resolved making the time for fulfillment long and arduous. In contrast, the problem identification module 144 determines any problem that may occur, regardless of when it occurs in the process, in fulfilling the prescription. The problem identification module 144 is coupled to access and retrieve data from the drug database 128. The problem identification module 144 uses the historical patient information, prescription history information, clinical information, prescriber outreach history, and drug information stored in the drug database 128, as described above with reference to FIG. 1, to identify potential problems that may occur during fulfillment of the prescription received. While the problem identification module 144 will now be described with regard to a specific set of problems, it should be understood that the problem identification module 144 may be modified to include additional problems other than those specifically identified below. Additional problems are within the scope of the operation of the problem identification module 144. For example, the problems may include patient cost, rejection by third parties such as insurers, whether the drug is in stock or currently available, whether the drug is subject to an insurer exclusion, whether the patient wants a brand-name drug versus a generic equivalent drug, and/or whether there is an operational exclusion.

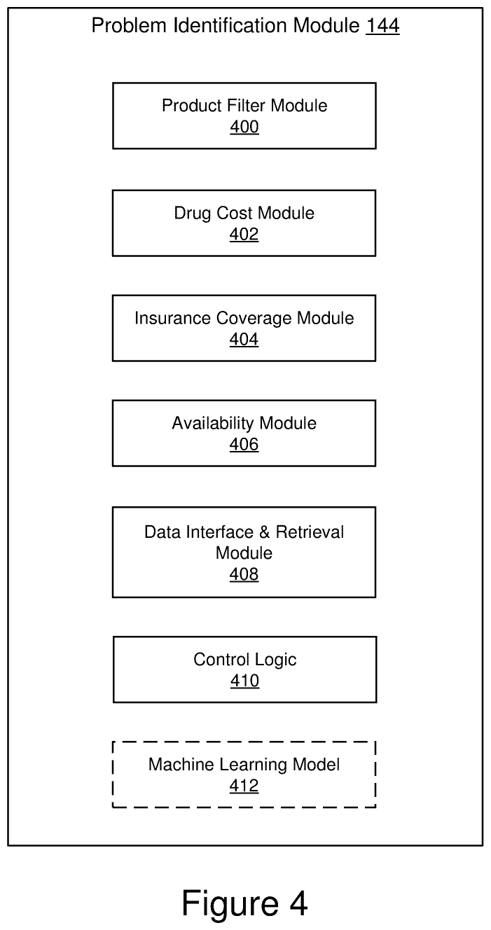

Referring now also to FIG. 4, the problem identification module 144 comprises a product filter module 400, a drug cost module 402, an insurance coverage module 404, an availability module 406, a data interface & retrieval module 408, a control logic 410 and optionally a machine learning model 412 in some implementations.

The product filter module 400 may be steps, processes, functionalities, software executable by a processor or a device including routines for excluding one or more products (e.g., prescriptions) from additional processing by the prescription optimization system 140. The problem identification module 144 may be configured to exclude certain product offerings. The product filter module 400 is coupled to the prescription intake module 142 to receive the prescription or other product that is to be filled by the pharmacy. The product filter module 400 advantageously allows additional processing by the prescription optimization system 140 to be omitted or skipped for selected prescriptions. For example, if the prescription is for a specialty drug (e.g., drug made specifically for patients by the pharmacist), a compound drug (e.g., compound drugs that contain multiple ingredients), or an immunization, the product filter module 400 will identify or mark the product so that the prescription optimization system 140 does not perform further processing such as identifying therapeutic alternatives and the next best action, but instead pass the received prescription on for normal fulfillment or rejection. Similarly, other products may be excluded such as canes and walkers, diabetic test strips, or COVID tests. The product filter module 400 can be configured to exclude items at a global system control level. In some implementations, the product filter module 400 includes one or more lists of prescriptions to compare with the prescription received from the prescription intake module 142. The product filter module 400 compares the drug in the received prescription to drug(s) on one or more list(s) for match. If a match is found, then the prescription is excluded from further processing by the prescription optimization system 140. In some implementations, the product filter module 400 maintains a first list of drugs excluded based on pharmacy criteria, a second list of drugs excluded by third parties, and a third list of drugs based upon policy issues. For example, the policy may be to never preclude a patient from getting a vaccine, so drugs related to immunizations are excluded from processing by the prescription optimization system.

The drug cost module 402 may be steps, processes, functionalities, software executable by a processor or a device including routines for determining whether the cost for a drug is above a threshold for the patient. The information received by the data interface and retrieval module 408 is provided to the drug cost module 402 so it can determine how much a particular drug or medicine will cost. In general, the drug cost module 402 identifies three types of cost problems: whether the drug will be of high cost to the patient, whether the drug is a high-cost drug for the pharmacy, or whether a coupon is available for the drug. In some implementations, the drug cost module 402 determines whether the prescribed drug is prohibitively expensive for the patient. In some implementations, the drug cost module 402 accesses the drug database 128 to determine how much the patient will have to pay in this instance for the prescribed drug. For example, the drug cost module 402 determines the cost of the prescribed drug, whether the patient has insurance, what the patient's co-pay for this particular drug would be, and what the patient has paid historically for this particular drug if at all. In some implementations, the drug cost module 402 interfaces with the data interface and retrieval module 408 to obtain real-time information about drug costs, claim coverage, and other factors that may affect the cost of a particular drug to the patient. The drug cost module 402 then determines whether the price for the prescription in this instance is greater by a threshold than the historical cost the patient has paid for the drug. For example, the drug cost module 402 may determine whether the drug will be considered expensive by the patient based on the patient's average co-pay. For example, the drug cost module 402 may also analyze a history of the patient including one or more of an average patient copay, a previously prescribed therapeutic previously tried by the patient, and an allergy profile of the patient. The drug cost module 402 may also determine whether the prescribed drug is on a list of high-cost drugs. If so, that problem may be identified by the drug cost module 402 in cases where the drug has a very high cost and the pharmacy wants to avoid issues such as excess inventory, excess returns by patients because of cost, a frustrating customer experience, and loss due to processing by the pharmacy. There may be additional problems related to cost by virtue of a coupon for the prescribed drug being available not available. A table of example cost type problems is shown below in Table 1.

TABLE 1

| Problem type | Problem |
|---|---|
| Cost | High Patient Cost: plus or minus a certain percentage of |
| | the patient's average co-pay. |
| Cost | High Patient Cost: minus a dollar amount of the patient's |
| | average co-pay. |
| Cost | High Patient Cost: co-pay greater than any price per paid |
| | for any prescription by this patient. |
| Cost | High Patient Cost: higher than the fixed dollar amount |
| | ever paid by this patient. |
| Cost | High Patient Cost: higher than a fixed dollar amount (no |
| | history for patient). |
| Cost | High-Cost Drug: Drug is very likely to go to waste |
| | because it's high cost and high likelihood of being returned |
| | to stock, also low margin. |
| Cost | Coupon: Not available. |
| Cost | Coupon: Patient or drug does not meet criteria. |
| Cost | COUPON: Expired. |

The insurance coverage module 404 may be steps, processes, functionalities, software executable by a processor, or a device including routines for determining whether there are any insurance coverage problems associated with the particular prescribed drug. The insurance coverage module 404 determines any insurance coverage problem as well as one or more steps to resolve the coverage problem. The insurance coverage module 404 is coupled to receive and send information to the drug database 128, the PBM system 114, and the pharmacy computing device 108. In some implementations, the insurance coverage module 404 determines whether the prescription is covered by insurance by processing the adjudication information related to the prescription that is provided by the prescription intake module 142. The insurance coverage module 404 may also determine when a prescription is not covered by a benefit plan of the patient but may be covered by a third-party. In some implementations, the insurance coverage module 404 may communicate with the pharmacy computing device 108 or the PBM system 114 to determine whether the prescription is covered by the patient's plan. These sources of information about insurance coverage may also be accessed by the insurance coverage module 404 to determine any other issues associated with insurance such as a different amount of co-pay or a required therapeutic alternative, as well as steps provided by the plan to resolve insurance coverage problems. In some implementations the insurance coverage module 404 is coupled to provide the determined problem and the steps to resolve it to the next best action module 150 for further processing. A table of example insurance type problems is shown below in Table 2.

TABLE 2

| Problem type | Problem |
|---|---|
| Insurance | Plan Coverage |
| Insurance | Coupon Rules |
| Insurance | Refill Too Soon |
| Insurance | Quantity To Dispense - e.g., only allowed to dispense 30 |
| | but dispensing 90 |
| Insurance | Third party rejection resolution (TPRR) - unpaid claim |
| Insurance | TPRR - third-party with code - system/software rejection |
| Insurance | TPRR - prior authorization needed |
| Insurance | TPRR - noncovered rejection - product not covered |
| Insurance | TPRR - noncovered rejection - drug not on formulary list |

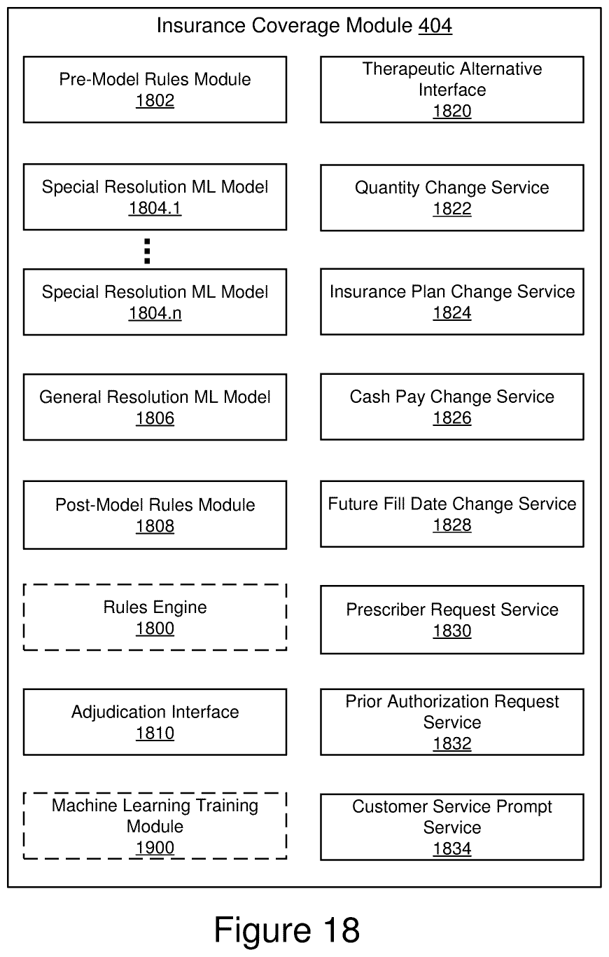

In some implementations, the insurance coverage module 404 may include a plurality of rules sets and machine learning modules configured to automatically resolve insurance adjudication rejections, either prior to claim submission through the PBM system 114 or in response to a claim rejection from an insurance adjudication system. For example, the insurance coverage module 404 may process the prescription information to identify problems, initiate next best actions to change one or more parameters, and automatically submit or resubmit an insurance claim for the prescription. An example implementation is further described with regard to FIG. 18 below.

The availability module 406 may be steps, processes, functionalities, software executable by a processor, or a device including routines for determining pharmacy operation problems or patient problems. The availability module 406 is coupled by the data interface and retrieval module 408 to the pharmacy computing device 108, the drug database 128, and the electronic medical record system 126. The availability module 406 receives pharmacy operations data from the pharmacy computing device 108, for example, information about the availability of specific drugs, additional authorizations or requirements for fulfillment, or any other pharmacy operations issues that may cause a delay or inability to fulfill a prescription. In some implementations, the availability module 406 is coupled receive real-time inventory information about the prescribed drug. The availability module 406 also determines whether a particular drug or medicine is in stock at a given pharmacy, when it will be available, or what pharmacies it is currently available at. For example, this information prevents the prescription optimization system 140 from attempting to fulfill a particular drug if it is not in stock, not available, or not provided by a particular pharmacy. Similarly, the availability module 406 is coupled to the drug database 128 and the electronic medical record system 126 to receive information about the patient that may raise issues related to the prescription being filled. For example, the availability module 406 may retrieve the patient's fill history or other information about the patient such as allergies or current prescriptions to determine drug interaction issues or problems that may be presented because of fulfillment of the current prescription. The availability module 406 receives and processes that information to determine any availability problems that may be encountered during fulfillment. The availability module 406 is coupled by the data interface and retrieval module 408 to provide the availability problems to the other components of the prescription optimization system 140 under the direction and control of the control logic 410. A table of example availability problems including pharmacy operation problems and patient problems is shown below in Table 3.

TABLE 3

| Problem type | Problem |
|---|---|
| Availability: Pharmacy | Backordered Prescription - need to check if |
| | geography affects dispensing store |
| Availability: Pharmacy | Insufficient Stock - quantity available to dispense |
| | (QAD) |
| Availability: Pharmacy | Insufficient Stock - store marked a drug as out of |
| | stock |
| Availability: Pharmacy | Insufficient Stock - nonpreferred drug |
| Availability: Pharmacy | Insufficient Stock - prescriber contact is set to no |
| | contact |
| Availability: Pharmacy | Insufficient Stock - prescriber request not needed |
| Availability | Insufficient Stock - prescriber request previously |
| | denied |
| Availability: Pharmacy | Out Of Stock |
| Availability: Pharmacy | Preorder |
| Availability: Pharmacy | Dispense As Written (DAW) - Prescriber |
| Availability: Patient | Dispense As Written (DAW) - Patient |
| Availability: Patient | Patient allergic to prescribed drug |
| Availability: Patient | Drug interaction problem with other current |
| | medications of patient |
| Availability: Patient | Fill history and rejections of patient |

The data interface and retrieval module 408 may be steps, processes, functionalities, software executable by a processor, or a device including routines for retrieving or receiving information from the other components of the system 100 that are needed to determine any problems that may be encountered during fulfillment of a prescription. For example, the data interface and retrieval module may assemble one or more parameter sets selected from prescription records, patient records, insurance records, and other data sources. The data interface and retrieval module 408 is coupled to the other components of the system 100 shown in FIG. 1. The data interface and retrieval module 408 is also coupled to the other components of the problem identification module 144. In some implementations, the data interface and retrieval module 408 receives real-time information from the other systems. For example, the pharmacist or pharmacy technician may input data related to or modifications to the prescription. The data interface and retrieval module 408 is coupled to the pharmacy computing device 108 to receive or retrieve this information. The patient may input comments or selections using the patient computing device 120 and the data interface and retrieval module 408 is coupled to receive this information. The data interface and retrieval module 408 is also coupled to the PBM system 114 or the pharmacy computing device 108 to receive claim and insurance information. Essentially, the data interface and retrieval module 408 is also coupled to receive real-time claim, cost, inventory and prescription data from these other systems shown in FIG. 1. The data interface and retrieval module 408 is coupled to send this real-time and other data it receives to the other components of the problem identification module 144.

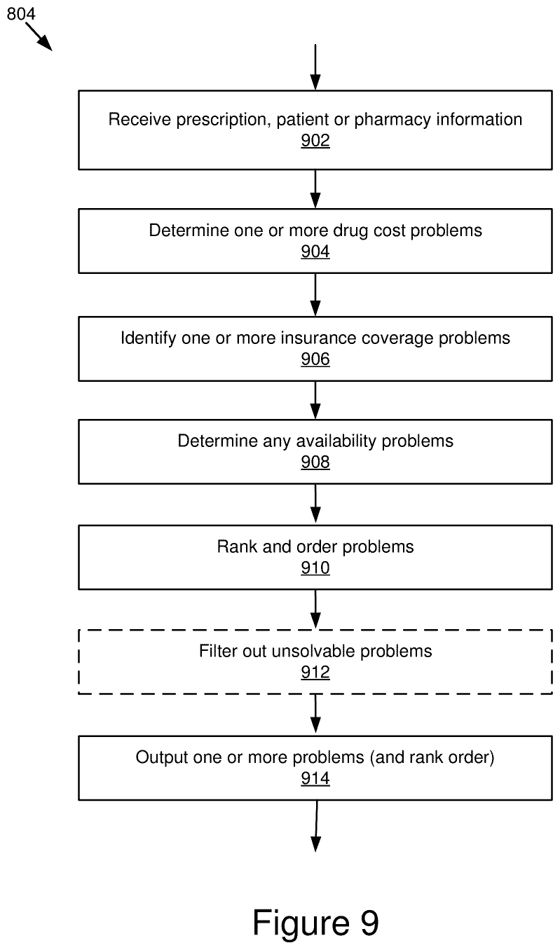

The control logic 410 may be steps, processes, functionalities, software executable by a processor, or a device including routines for controlling the operation of the problem identification module 144. The control logic 410 is coupled to the other components of the problem identification module 144 to control them. The operation of the problem identification module 144, and similarly, the control logic 410 will be described below with reference to the flowchart of FIG. 9. In one implementation, the control logic 410 identifies the problems and ranks them according to an assigned priority, then sends all the identified problems to the therapeutic alternative recommendation module 146 and the next best action module 150 for further processing as depicted in FIG. 3. In some implementations, the problem identification module 144 is a rule set to determine the problems that may occur in fulfillment of a given prescription such as with the control logic 410.

In some implementations, the control logic 410 may to determine what problem(s) exist, what problem(s) are potentially resolvable, and what problem(s) are not resolvable. Based on those determinations, the control logic 410 proceeds to resolve the most important resolvable problem so that a prescription may be fulfilled. For example, the control logic 410 implements a set of rules, which identifies what all the problems are. The control logic 410 then determines whether any of the problems are unresolvable. If so, the control logic 410 does not attempt to fix the resolvable problem because an unresolvable problem exists. For example, if a particular drug is not covered by insurance, then determining whether there is a therapeutic alternative is a potential solution to that problem. On the other hand, if neither the particular drug nor its therapeutic alternatives are covered by insurance, there is no reason to resolve whether the drug is out of stock. In this instance, whether the drug is out of stock will not matter because a particular patient cannot get it on their insurance plan. So, the primary problem becomes that the drug is not covered by insurance and that is the problem identified by the control logic 410 for resolution. In some implementations, the control logic 410 may also sort and rank the identified problems into a set or list. The control logic 410 may use a variety of different criteria to rank and sort the identified problems. For example, those criteria may include severity of the problem, time delay caused by the problem, likelihood of resolution of the problem, reoccurrence of the problem, etc. This ranked set or list problems may be provided to the next best action module 150. In some implementations, the control logic 410 provides only a single problem to the next best action module 150.

In some implementations, the control logic 410 may also determine whether one or more therapeutic alternatives should be identified, and sends a request to the therapeutic alternative recommendation module 146 including the prescription to make such an identification. The problem identification module 144 may need to identify a therapeutic alternative to address any problems that it identifies including, but not limited to, cost, insurance coverage, drug availability, or other reasons. In such an implementation, the control logic 410 is coupled for communication with the therapeutic alternative recommendation module 146 to send or trigger the generation of a list of therapeutic alternatives for the prescription received by the problem identification module 144. In some implementations, the problem identification module 144 merely provides the problems to the therapeutic alternative recommendation module 146, and the therapeutic alternative recommendation module 146 determines whether therapeutic alternative should be identified, or not, based on the problems that are received by it from the problem identification module 144. In yet another implementation, the problems are provided by the problem identification module 144 to the next best action module 150, and the next best action module 150 cooperates with the therapeutic alternative recommendation module 146 to determine whether a therapeutic alternative should be determined and for which problems.

The machine learning model 412 may be steps, processes, functionalities, software executable by a processor, or a device including routines for determining which problems to provide to the therapeutic alternative recommendation 146 and the next best action module 150. The machine learning model 412 may also rank order the problems based on importance. The machine learning model 412 is shown with dashed lines to indicate that it is optional. In some implementations, the problem identification module 144 uses the machine learning model 412 to determine the problems that may occur in fulfillment of a given prescription. In general, the relationships learned may involve training from pharmacist, pharmacist technicians, users, experts, employees, automated data feeds from third parties, or some combination thereof. The machine learning model 412 may be geometric systems like nearest neighbors and support vector machines, probabilistic systems, evolutionary systems like genetic algorithms, decision trees, neural networks, associated with decision trees, Bayesian inference, random forests, boosting, logistic regression, faceted navigation, query refinement, query expansion, singular value decomposition and the like. The machine learning model 412 may use supervised learning, semi-supervised learning, or unsupervised learning for building and training the machine learning systems based on the type of data available and the particular machine learning technology used for implementation. In some implementations, one or more machine learning models may be used to determine the problems similar to the drug cost module 402, the insurance coverage module 404, and the availability module 406 described above.

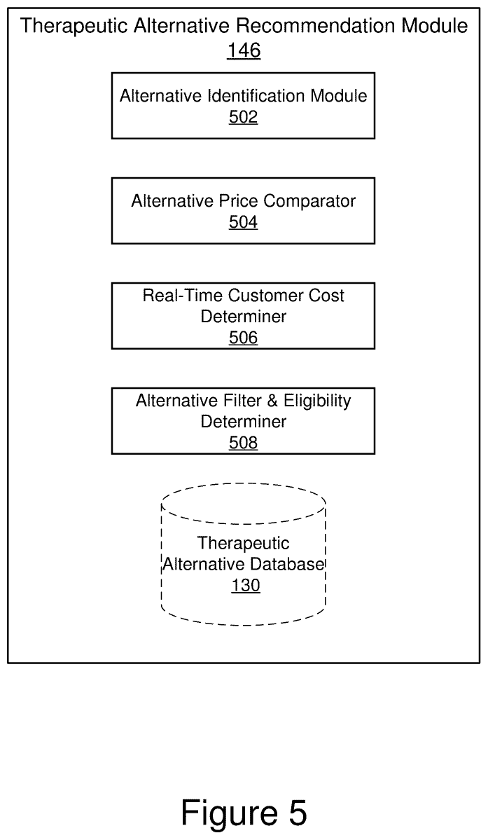

Referring back to FIG. 3 and now also to FIG. 5, the therapeutic alternative recommendation module 146 according to some implementations is described. The therapeutic alternative recommendation module 146 may identify one or more therapeutic alternatives to the prescription for the patient and recommend one or more drugs as a substitute for the prescription received by the prescription intake module 142. The therapeutic alternative recommendation module 146 is coupled to and configured to access a therapeutic alternative database 130. In some implementations, the therapeutic alternative database 130 is part of the therapeutic alternative recommendation module 146 has shown in FIG. 5. However, the therapeutic alternative database 130 may also be coupled to the network 102 as depicted in FIG. 1, and the therapeutic alternative recommendation module 146 accesses the therapeutic alternative database 130 via the network 102. As shown in FIG. 3, the therapeutic alternative 146 is coupled to receive a request from the problem identification module 144 for a therapeutic alternative for a given prescription and provide one or more therapeutic alternatives to the next best action module 150 in response. Preferably, the therapeutic alternative recommendation module 146 is able to identify a therapeutically equivalent drug at a lower cost to the patient.

As will be described in more detail below with reference to FIG. 5, the therapeutic alternative recommendation module 146 uses several factors to determine and recommend a therapeutic alternative. As shown in FIG. 5, in some implementations, the therapeutic alternative recommendation module 146 comprises an alternative identification module 502, an alternative price comparator 504, a real-time customer cost determiner 506, and an alternative filter and eligibility determiner 508. It should be understood that one or more additional factors beyond those described below with reference to FIG. 5 may be used in determining a therapeutic alternative.

The alternative identification module 502 may be steps, processes, functionalities, software executable by a processor, or a device including routines for identifying one or more therapeutic alternative drugs to the prescription for the patient. The alternative identification module 502 is coupled to receive information about the patient and the prescription, for example, by accessing the drug database 128. The alternative identification module 502 is also coupled to receive information from the therapeutic alternative database 130. The alternative identification module 502 uses the prescription information and searches the therapeutic alternative database 130 for therapeutic alternatives. The alternative identification module 502 accesses the therapeutic alternative database 130 to ensure that the prescription is mapped to a therapeutic alternative in the database 130. The therapeutic alternative database 130 includes additional information that is checked to ensure that the one or more therapeutic alternatives identified are properly equivalent to the prescription. For example, the alternative identification module 502 performs comparisons such as a formulary check, a day supplies check, insurance eligibility, drug interaction, etc. It should be understood that the alternative identification module 502 may consider various other factors in determining which drugs qualify as a therapeutic alternative in addition to those listed above. For example, the therapeutic alternative database 130 may exclude certain alternatives because they require a consultation with a physician. Once the alternative identification module 502 has identified one or more therapeutic alternatives, it sends the one or more therapeutic alternatives to the alternative price comparator 504, real-time customer cost determiner 506 and the alternative filter and eligibility determiner 508 for further processing as will be described below. In some implementations, the identified one or more therapeutic alternatives may be stored in cache for use by the other components of the therapeutic alternative recommendation module 146 and the next best action module 150.

The alternative price comparator 504 may be steps, processes, functionalities, software executable by a processor, or a device including routines for comparing the prices of the prescription and the one or more therapeutic alternatives. The alternative price comparator 504 is coupled to receive the prescription for the patient and the one or more therapeutic alternatives from the alternative identification module 502. The alternative price comparator 504 is also coupled to the real-time customer cost determiner 506 to receive the cost for the patient for the prescription as well as the one or more therapeutic alternatives. The alternative price comparator 504 receives this information and generates a ranked list including the prescription and the one or more therapeutic alternatives ranked by price. The alternative price comparator 504 preferably list the drugs from lowest cost to highest cost. It should be understood that in some implementations the alternative price comparator 504 determines the price of each drug for a particular patient. This includes a determination of whether the drugs are covered by insurance and the amount of co-pay that will be required by the patient. The output of this ranked list is provided by the alternative price comparator 504 to the alternative filter and eligibility determiner 508.

The real-time customer cost determiner 506 may be steps, processes, functionalities, software executable by a processor, or a device including routines for determining the cost of the prescription as well as the therapeutic alternatives determined by the alternative identification module 502. The real-time cost determiner 506 is coupled to the alternative identification module 502 to receive one or more therapeutic alternatives for the prescription. The real-time cost determiner 506 is also coupled to alternative price comparator 504 to provide the cost for this patient for the prescription and the one or more therapeutic alternatives. In some implementations, the real-time customer cost determiner 506 may be configured to identify patient copays for the prescription and the one or more therapeutic alternatives. For example, the real-time customer cost determiner 506 may be configured to generate a respective hypothetical prescription insurance claim for the prescription and the one or more therapeutic alternatives; and receive respective patient copays in response to the hypothetical prescription insurance claims for the prescription and one or more the therapeutic alternatives specified by the alternative identification module 502. Further, the real-time customer cost determiner 506 may store each of the respective patient copays for each of the prescription and the therapeutic alternatives for the treatment of the patient in the drug database 128. In some implementations, the real-time customer cost determiner 506 also checks one or more of the prescription attributes such as days supply, eligibility, formulary check, etc. to ensure that the cost comparison performed by the alternative price comparator 504 is comparing drugs with similar attributes.

The alternative filter and eligibility determiner 508 may be steps, processes, functionalities, software executable by a processor, or a device including routines for determining the one or more therapeutic alternatives to provide to the next best action module 150 and ultimately to the problem identification module 144. The alternative filter and eligibility determiner 508 is coupled to the alternative identification module 502 to receive the list of therapeutic alternatives identified. The alternative filter and eligibility determiner 508 is also coupled to the alternative price comparator 504 to receive a list of therapeutic alternatives and their associated costs. The alternative filter and eligibility determiner uses the cost information to determine which one or more therapeutic alternatives to output to the next best action module 150 and the problem identification module 144. In some implementations, the alternative filter and eligibility determiner 508 may filter or mark ineligible certain therapeutic alternatives; and thereby, remove them from the one or more therapeutic alternatives that are output. Such ineligibility may be based upon the prescription being a compounded medication, a specialty medication, or an immunization. Still further, the alternative filter and eligibility determiner 508 may be configured to deny a determination of therapeutic alternative based upon prior rejections by a pharmacist to approve the therapeutic alternative, denial by the prescriber of the therapeutic alternative or rejection of the patient of the therapeutic alternative. In some implementations, the alternative filter and eligibility determiner 508 filters out therapeutic alternatives based on the particular patient such as their allergies, past ineffective use, or interaction with other drugs they are taking. The filtered one or more therapeutic alternatives and the corresponding price to the patient are output by the alternative filter and eligibility determiner 508.

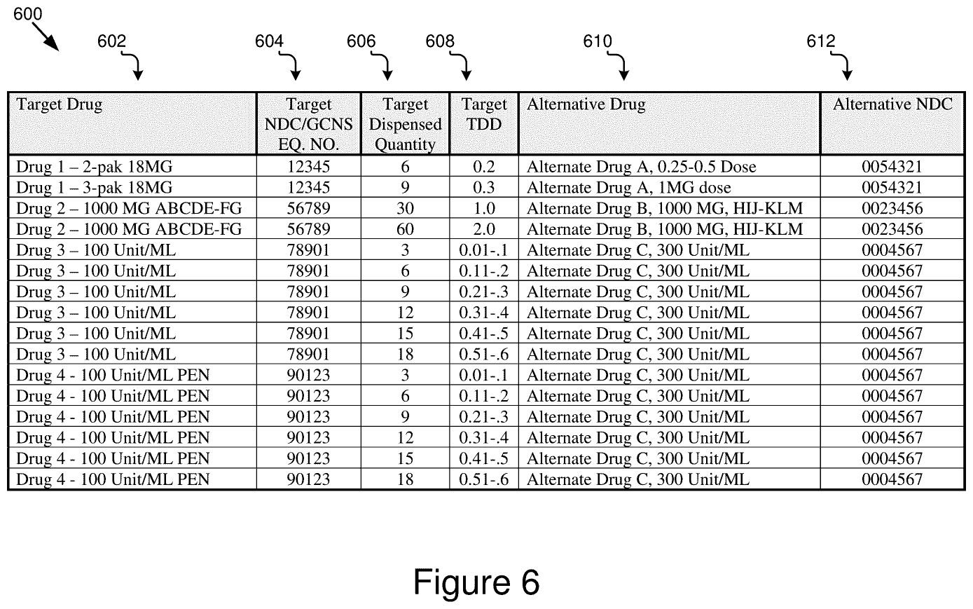

FIG. 6 illustrates an example therapeutic alternative list 600 of the therapeutic alternative database 130 in accordance with some implementations. The therapeutic alternative list 600 may be formed based upon clinical mapping of the drug identified in the prescription for the treatment of the patient. The therapeutic alternative list 600 may be populated using potential viable alternatives that achieve a similar therapeutic outcome as the drug specified in the prescription. Alternatives may be in the form of generic drugs or other drugs identified using a collection of clinical knowledge. The drug identified in the prescription for the patient may also be indexed using NDC or other indexing information. By way of example, the therapeutic alternative list 600 includes a target drug 602, a target drug NDC or other index 604, a target dispense quantity 606, a target drug total daily dose 608, and alternative drugs 610 with an alternative NDC 612. As noted in the example therapeutic alternative list 600, a target drug 552 may be listed one or more times depending upon the number of alternative drugs 562 that have been identified and stored in the therapeutic alternative list 600.

Referring back to FIG. 3, the output of the therapeutic alternative recommendation module 146 is provided to the next best action module 150. In some implementations, the next best action module 150 is particularly advantageous because it, like the problem identification module 144, identifies and considers any problem that may occur in the fulfillment process through to completion in determining the next best action. Information output by the problem identification module 144 is also provided to the next best action module 150 as shown. In some implementations, the problems described above that were generated by the drug cost module 402, the insurance coverage module 404, and the availability module 406 of the problem identification module 144 are sent to the next best action module 150, and these problems are used to determine possible actions and the next best action. For example, the problems identified such as those described above with reference to Tables 1-3 can be sent by the problem identification module 144 to the next best action module 150.

In some implementations, the problem identification module 144 generates data elements, and these data elements are provided to the next best action module 150. The data elements provided by the problem identification module 144 to the next best action module 150 can be organized by category, and Table 4 below shows how the data elements are used by the next best action module 150 to avoid problems and optimize the fulfillment process. The uses described below in Table 4 are implemented by operation of the rules logic 708 as will be described below. It should be understood that the data elements and uses listed in Table 4 below are merely examples and that there may be additional data elements with new uses or additional uses for the existing data elements. For example, the data elements may be partitioned into categories such as a workflow category, a prescription category, and a patient category. This categorization is used by the rules logic 708 in selecting a next best action. Data elements in the workflow category relate to how the pharmacy operations are performed, pharmacist or pharmacy technician actions, interaction with third-party payors, and how tasks proceed through the fulfillment workflow at the pharmacy. Data elements in the prescription category relate to information about the particular prescription itself such as eligibility for processing, status of the prescription as out of stock or on backorder, location at different pharmacies, requiring preorder, or a build of the coupons. Data elements in the patient category relate to information about the patient such as allergies, communication preference, other prescriptions the patient is already taking, etc. As will be described below, the next best action module 150 analyzes this information to determine a next best action for the particular patient and prescription. These example data elements are shown in Table 4 below.

TABLE 4

| Category | Data Elements | Use by Next Best Action Module 150 |
|---|---|---|
| Workflow | Pharmacist rejected | Prevent a loop on edit to not retrigger the same |
| | prescriber requests | opportunity when a prescription is rejected for |
| | (PR) | reason “PR not needed.” |
| Workflow | Denied prescriber | Prevent triggering therapeutic alternative |
| | responses | requests on a prescription with a recent |
| | | denial of alternatives. |
| Workflow | Prescriber contact | Prevent outbound PRs to prescribers on |
| | preference | channels with reported service issues. |
| Workflow | Third party rejection | Provide alternatives in cases where TPRR could |
| | resolution (TPRR) | not be resolved, and Third party could not |
| | automation level | provide real-time adjudication results. |
| Workflow | Real-time claim | Understand if the reject can be resolved by the |
| | adjudication result | user before considering alternatives. |
| Rx | Ineligible prescriptions | Prevent processing of compounds, specialty |
| | or other over the counter | drugs, and immunizations. |
| | items | |
| Rx | Quantity available to | Determine current out-of-stock and backorder |
| | dispense | status. |
| Rx | Quantity available at | Suggest availability at another pharmacy |
| | other close pharmacies | in close proximity. |
| Rx | Backordered NDC list | Provide differentiated fill options when a |
| | | product has a known supply chain shortage. |
| Rx | Preorder and Central Fill | Provide accurate messaging to patients about |
| | program-level indicators | delay and or additional information needed. |
| Rx | Drug coupon eligibility | Calculate cost using coupon. |
| | | Suspend coupon action until evaluation is |
| | | complete. |
| Patient | Patient average copay | Used to define or redefine whether a product is |
| | | expensive from patient perspective. |
| Patient | Fill history of therapeutic | Indicative of whether patient will use therapeutic |
| | alternatives | alternatives. |
| | | Also used to prevent populating a prescriber |
| | | request with a therapeutic alternative that the |
| | | patient has already tried in the past. |
| Patient | Patient allergy profile | Eliminating suggesting therapeutic alternatives |
| | | to which the patient is allergic. |
| Patient | Patient current medications | Prevent suggesting therapeutic alternatives that |
| | | may cause drug interaction problems. |

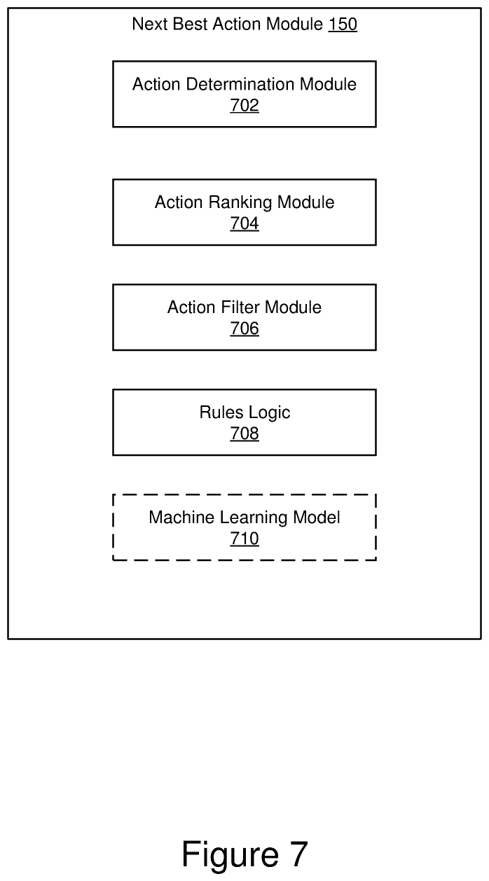

Referring now also to FIG. 7, a block diagram illustrating an example of a next best action module 150 in accordance with some implementations is shown. The next best action module 150 generates a next best action for a prescription. The next best action module 150 comprises an action determination module 702, an action ranking module 704, an action filter module 706, and rules logic 708. Similar to the problem identification module 144, the next best action module 150 may optionally include a machine learning model 710. The action determination module 702 determines one or more actions to be taken based upon the problems identified by the problem identification module 144. The action determination module 702 provides these actions to the action ranking module 704. The action ranking module 704 ranks the different actions that can be taken, and prioritizes them based on a variety of factors. The ranked list of actions is then provided by the action ranking module 704 to the action filter module 706. The action filter module 706 removes any actions from the ranked list based on factors that may require certain actions to be removed. The action filter module 706 provides the filtered list of actions that can be taken to the rules logic 708. Finally, the rules logic 708 is applied to the filtered list of actions and one action is selected as the next best action for execution. This one selected action is sent by the rules logic 708 to the pharmacy computing device 108, in particular the action execution module 152, to perform the selected action. In some implementations, the functions and activities described above with reference to components of the next best action module 150 can be performed by the machine learning model 710.

The action determination module 702 may be steps, processes, functionalities, software executable by a processor, or a device including routines for determining the possible actions for a given prescription. The action determination module 702 is coupled to the problem identification module 144 to receive a list of problems associated with a prescription. In some implementations, each problem and associated action are identified with an exception label. For example, exception labels may include: 1) insufficient stock or not in stock; 2) requires a special order; 3) drug not being covered by insurance; 4) potentially being too expensive (e.g. being beyond a threshold of patient adjustable cost); etc. The action determination module 702 also determines an action that can be taken to resolve a given problem or address an exception label.

Some example actions may include:
putting the drug into queue to be ordered (e.g., an inventory queue);
determine geography affected by back order, compare to pharmacy location and either communicate problem to patient or complete unaffected fill;
sending out a special order;
requesting an insurance coverage exemption;
requesting prior authorization from a TPRR;
determine a therapeutic alternative drug;
fill prescription the prescription despite problem (perhaps based on a patient's fill history or other factors);
communicate with the prescriber;
send prescriber request for a different drug therapy or suggesting a therapeutic alternative;
send prescriber request for confirmation of a DAW;
send prescriber request for product backordered/unavailable;
communicate with the pharmacist;
request pharmacist performs a specific action;
display inventor exception to pharmacist;
communicating with patient (e.g., via text, email phone or other) about status, problem or offer and confirmation of a selection;
communicate with patient about alternative actions;
communicate with patient re preorder and inform about availability, delay or status;
communicate with patient re how they would like the problem resolved;
communicate with patient re DAW and lower cost therapeutic alternatives;
confirm patient's DAW and interest in therapeutic alternatives;
transfer the prescription to another pharmacy;
review patient fill history, and based on fill history do nothing, fill prescription, or take other action in this list; or
doing nothing.

Once the action determination module 702 identifies the problems and corresponding actions to address the problems for a given prescription, the action determination module 702 sends the list of problems and corresponding actions to the action ranking module 704.

The action ranking module 704 may be steps, processes, functionalities, software executable by a processor, or a device including routines for ranking or prioritizing actions that can be taken to address a problem with a prescription. In some implementations, the problems with the greatest impact toward fulfilling the prescription will be rank the highest. If only a single problem and a corresponding action is identified, the action ranking module 704 sends the single problem and the identified solution for as a next best action. However, if there is even a single problem, that single problem may have multiple different actions that can be taken to resolve the problem. The action ranking module 704 will select the action which has the highest impact and send the problem and that selected action forward as the next best action. When there are multiple problems with a given prescription, the action ranking module 704 must select among different problems and different actions. The action ranking module 704 again selects the action which is going to have the greatest impact toward fulfilling the prescription. In one example case, some prescriptions have several problems that are determined to be more severe, like a prescription is prohibitively expensive, not currently in stock and not approved for insurance coverage. In such a case, the recommended action to be performed may be prescriber outreach. For this case, the action determination module 702 identifies an action of intercepting the prescription and asking the provider for an alternative so that alternatives can be determined before the patient even is aware that there is a problem. The next best action module 150 determines that there is a problem with a drug being very expensive, determines a lower cost option for the patient, with the provider's permission, and then notifies the patient that a problem was identified with the original script, and the prescription optimization system 140 reached out to the physician to obtain a lower cost approved medication. The prescription is filled, and the only action remaining is to advise the patient that a lower cost alternative was filled and at the point of sale they can make a decision whether to accept the filled prescription or request that the original script be filled, despite the identified problems. Other factors that may be used to rank the actions can include resolvability of the problem, responsiveness of prescribers, pharmacists and patients, value of communication despite lack of resolution, strictness or flexibility of an insurance coverage provider, availability of the therapeutic alternative, effectiveness of therapeutic alternatives, time required for the action to resolve the problem, prescriber experience, patient experience, pharmacist experience, and other factors. In particular, for the prescriber experience and patient experience, the ranking may be configured to avoid many multiple requests so actions are not repeated where the prescriber or the patient has already considered the requests and denied or rejected them. In some implementations, the action ranking module 704 processes and evaluates past prescriber or patient behavior as part of ranking the actions. For example, the action ranking module 704 may review past therapies and reduce the rank for therapeutic alternatives if the patient has rejected them in the past to make sure therapeutic alternatives that they have already rejected or considered before are not presented again. In one implementation, the action ranking module 704 implements a ranking using a rule set where problems are ordered based upon their exception label. The priority in the rule set was developed based upon dependencies in the fulfillment process and the problems that if addressed early would have the greatest impact on the fulfillment process. Depending on the exception label assigned to a problem, the rule set processes the problems in a particular order. For example, the exception labels may be processed in the following order 1) third party rejection; 2) prior authorization; 3) high-cost hold; 4) backordered; 5) insufficient stock; 6) high patient pay; and finally, 7) DAW. In another implementation, the problem/action pairs are prioritized using a configurable hierarchy. In still other implementations, the action ranking module 704 uses a weighted average of any one or more of the above listed factors to generate a score for each problem/action pair. Then the problem/action pairs are ranked based upon the generated score and a list of problems/action pairs and their scores is output by the action ranking module 704 to the action filter module 706.

The action filter module 706 may be steps, processes, functionalities, software executable by a processor, or a device including routines for removing any actions from the ranked list from the action ranking module 704 based on a variety of different factors. Certain actions may be removed or be required to be based on different policy or regulatory reasons. Similarly, certain problems where the recommended action may adversely affect the patient, require an in-person consultation, or are highly likely to be negatively received by the patient may be removed as a problem/action pair from the ranked list. Similarly, the prescription alone may be the basis for an action being filtered out. Additionally, the action filter module 706 may provide redundancy with the product filter module 400 and remove actions related to compound drugs, specialty drugs or immunizations or it may provide that function in alternate implementations where the product filter 400 is omitted from the problem identification module 144. Likewise, the action filter module 706 may remove any prescription/action pairs where the patient may have an allergic reaction or may have drug interaction problems because of other drugs they are already taking.

The rules logic 708 may be steps, processes, functionalities, software executable by a processor, or a device including routines for selecting a next best action from all the actions evaluated by the next best action module 150. The rules logic 708 is coupled to the action filter module 706 to receive the list of filtered, ranked list of problem/action pairs. The rules logic 708 selects one action from the list of filtered, ranked list of problem/action pairs as the next best action for execution. In some implementations, the rules logic 708 outputs or presents a list of one or more actions ordered by ranking. The rules logic 708 is configured to provide the selected next best action or set of actions to the action execution module 152 of the pharmacy computing device 108. In one implementation, the rules logic 708 selects the action associated with the problem action pair that is ranked highest in the filtered ranked list. In other implementations, the rules logic 708 may include a rules engine configured to apply a more complicated rule set to determine the next best action based upon applying the rule set to the list of filtered, ranked list of problem/action pairs. In some implementations, the rule set may use a variety of factors and other information known about the patient, the prescription, the pharmacy, etc. to determine which of the problem/action pairs is going to most significantly increase the likelihood of fulfillment, reduce the time for fulfillment, or eliminate patient dissatisfaction. In some implementations, the rules logic 708 may perform alternate processes for comparison as to which action would be most effective to advance the prescription and the filling process.

The machine learning model 710 may be steps, processes, functionalities, software executable by a processor, or a device including routines for determining a next best action. In some implementations, the machine learning model 710 replaces the components of the next best action module 150 and receives information from the problem of identification module 144 and the therapeutic alternative recommendation module 146 and uses the received information to generate a next best action. In other implementations, the machine learning model 710 may be used in place of any one of the above components of the next best action module 150. In such other implementations, there may be a plurality of machine learning models 710, and each machine learning model receives the signals and generates the output as has been described above with reference to components of the next best action module 150. The machine learning model 710 may be geometric systems like nearest neighbors and support vector machines, probabilistic systems, evolutionary systems like genetic algorithms, decision trees, neural networks, associated with decision trees, Bayesian inference, random forests, boosting, logistic regression, faceted navigation, query refinement, query expansion, singular value decomposition and the like. The machine learning model 710 may use supervised learning, semi-supervised learning, or unsupervised learning for building and training the machine learning systems based on the type of data available and the particular machine learning technology used for implementation.

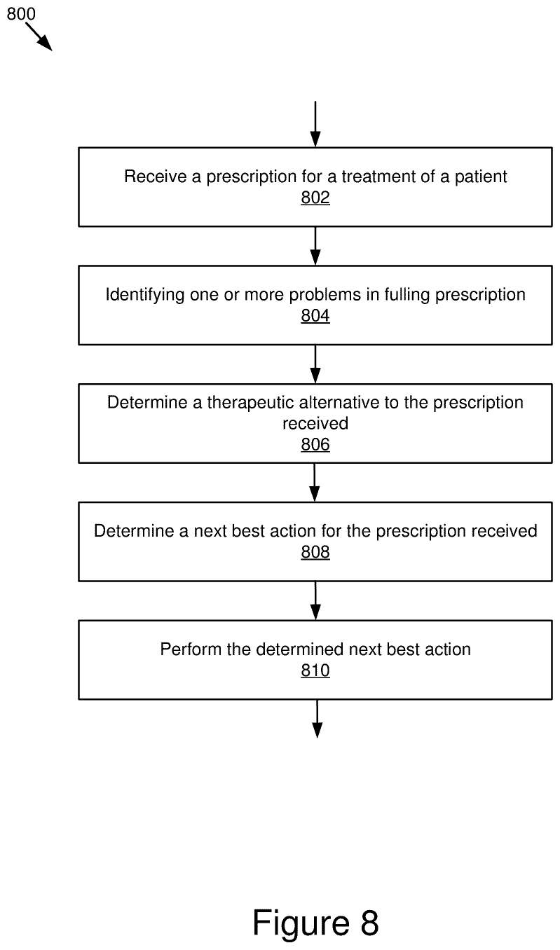

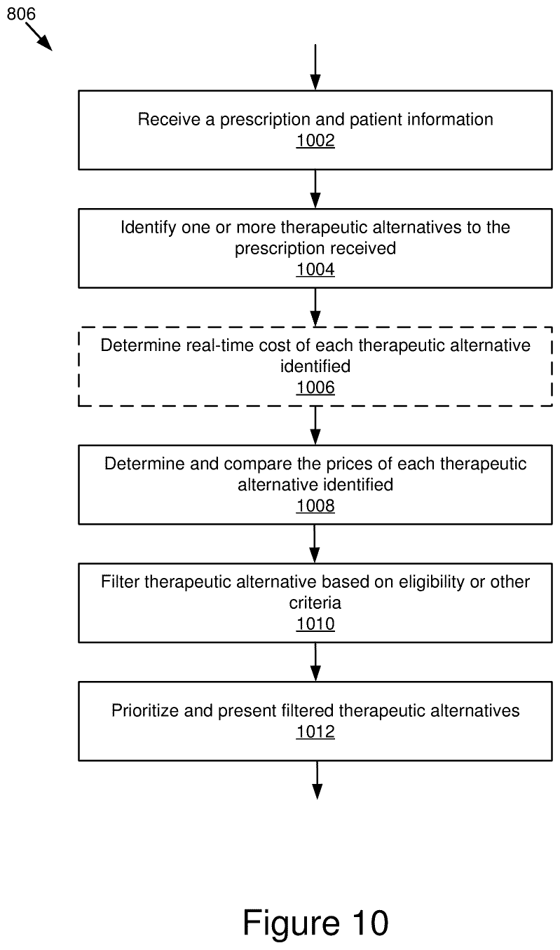

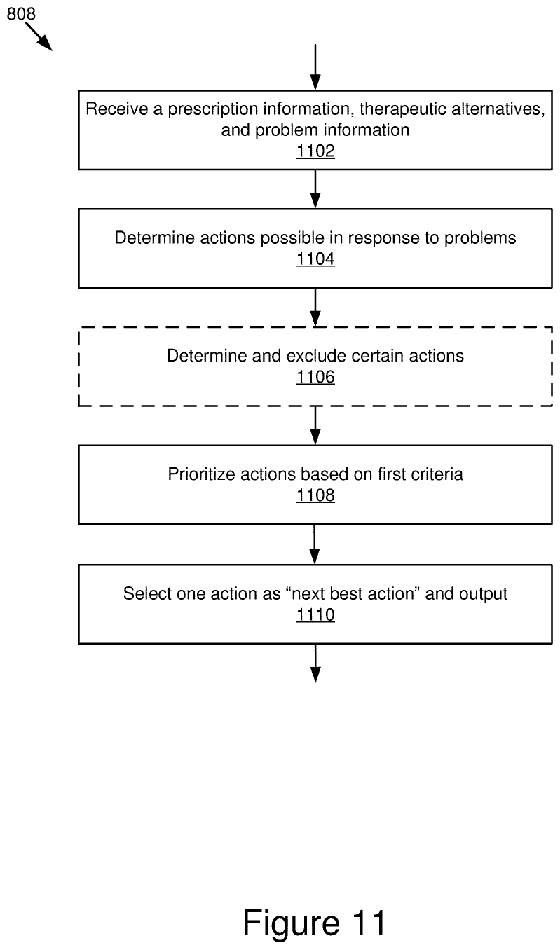

Referring now to FIG. 8, a method 800 for optimizing fulfillment of a prescription in accordance with some implementations is described. The method 800 begins by receiving 802 a prescription for a treatment of the patient. The prescription may be received by the pharmacy computing device 108, in particular the prescription intake module 142. The prescription intake module 142 collects information related both to the patient and the prescription and sends that information to the prescription optimization system 140. For example, a history of the patient may be analyzed including identifying an average patient copay, a previously prescribed therapeutic alternative that was previously tried by the patient, and/or an allergy profile of the patient. The method 800 continues by identifying 804 one or more problems in fulfilling or processing the prescription. This identification 804 is performed by the problem identification module 144 as described above and will be described in more detail below with reference to FIG. 9. Next, the method 800 determines 806 one or more therapeutic alternatives to the prescription received for the patient. Determining a therapeutic alternative is performed by the therapeutic alternative recommendation module 146 and will be described in more detail below with reference to FIG. 10. The method 800 continues by determining 808 a next best action to perform in order to fulfill the prescription in the most efficient and customer-oriented way. The determination 808 of the next best action can be performed by the next best action module 150. The next best action module 150 receives the one or more problems identified in block 804 and the one or more therapeutic alternatives determined in block 808, and uses them to determine the next best action. One example process for determining the next best action will be described below with reference to FIG. 11. The method 800 continues by performing 810 the next best action identified in block 808. In some implementations, the next best action is sent from the prescription optimization system 140 to the pharmacy computing device 108, and the next best action is executed by the action execution module 152. It should be understood that in the process of filling a prescription, the above steps of the method 800 may be performed repeatedly as each problem is identified and resolved until the prescription can be filled.

Referring now to FIG. 9, a method 804 for identifying a problem fulfilling or processing a prescription in accordance with some implementations is described. The method 804 begins by receiving 902 prescription information, patient information and/or pharmacy information. For example, the prescription information may be the drug being prescribed, the patient information may be the drugs the patient is taking or has taken in the past, insurance information may be which drugs are covered by insurance or alternative therapies approved, other pharmacy information may be back-order status. Next the method 804 determines 904 one or more drug cost problems. For example, the method 804 may determine whether the cost for filling the prescription is more than a threshold above what the patient has historically paid for the prescription, or what the system predicts the patient would be willing to pay based on past prescription costs. This step may be performed by the drug cost module 402 described above with reference to FIG. 4. The method 804 continues to identify 906 one or more insurance coverage problems. There may be a variety of insurance coverage problems including but not limited to whether the prescription is covered, whether a coupon is applicable, whether a third-party payor will reject the prescription, etc. In some implementations, this step 906 is performed by the insurance coverage module 404 as has been described above. Next, the method 804 determines 908 whether there is any availability problem with the prescribed drug. For example, the drug may be out of stock at a particular pharmacy, out of stock at the warehouse, temporarily unavailable because of supply chain issues, or unavailable or delayed for delivery for any variety of reasons. In some implementations, this step 908 is performed by the availability module 406 described above with reference to FIG. 4. In some implementations, the method 804 may also determine whether there are any availability issues associated with therapeutic alternatives that are recommended in place of the prescription. Next, the method 804 ranks and orders 910 the identified problems to produce a set or list of problems. As optionally shown with dashed lines in block 912, the method 804 may also filter out unsolvable problems. In some implementations, unsolvable problems may be provided with a lower or the lowest rank. Then the method 804 outputs 914 the one or more problems to the next best action module 150 or other systems. In some implementations, the set or list can be ranked or unranked. It should be understood, that the present disclosure allows identified problems to be filtered or excluded at either the problem identification module 144 or the next best action module 150. In some implementations, both modules 144, 150 may perform filtering or exclusion steps if redundancy is required.

Referring now to FIG. 10, a method 806 for determining a therapeutic alternative in accordance with some implementations is shown. The method 806 begins by receiving 1002 prescription and patient information. For example, a patient receives from a physician a prescription for one or more medications. As part of the prescription process, the physician may log into a physician computing device 104 and access an e-prescribing application 106 where the physician can enter the prescription and treatment information. In block 1002, the prescription is also processed for the treatment of the patient. The processing may be performed in prescription intake module 142 as described above. The prescription intake module 142 may also analyze a history of the patient including one or more of an average patient copay, a previously prescribed therapeutic alternative previously tried by the patient, and an allergy profile of the patient. In block 1004, one or more therapeutic alternatives to the prescription may be identified or mapped. The processing or mapping may be performed by the alternative identification module 502 as described above. The identification of a list of therapeutic alternatives may include accessing the therapeutic alternative database 130 which may be indexed based upon the prescription for the treatment of the patient, drug type, formulary equivalence, and other attributes. Further, the therapeutic alternative database 130 may also be indexed based upon a national drug code for the prescription for the treatment of the patient. Further, duplicates of therapeutic alternatives may also be rejected for efficiency. In some implementations, the method 806 may determine 1006 a real-time cost of each therapeutic alternative identified in block 1004. This block 1006 is optional so it is depicted in dashed lines. In block 1006, the real-time customer cost determiner 506 determines the cost of the prescription to this particular patient based upon the insurance coverage of the patient, the co-pay of the patient, the available inventory and cost of the prescription at the time and other real-time factors. This determination 1006 has been described above with reference to the real-time customer cost determiner 506. In the event step 1006 is not performed, the method 806 uses a schedule of standard or retail prices. The method 806 continues by determining and comparing 1008 the price for each therapeutic alternative identified in block 1004. This determination and comparison process 1008 has been described above with reference to the alternative price comparator 504. The method 806 continues to filter 1010 therapeutic alternatives based on eligibility and other criteria. For example, certain alternatives may be removed from the list based on whether they are covered by insurance or not. Additional therapeutic alternatives may be removed using other criteria, e.g., cost, medication type, or prior rejections by prescribers. Next, the method 806 prioritizes and presents 1012 a list of filtered therapeutic alternatives. The list of filtered therapeutic alternatives may be prioritized, reordered or ranked for optimization of the system based on a variety of different factors such as available inventory, pharmacy process, therapeutic effectiveness, efficacy for this patient, client copays, etc. In some implementations, a rule set is used to create the ranked list. Once the list of therapeutic alternatives has been prioritized, it is provided to the next best action module 150.

Referring now to FIG. 11, a method 808 for determining a next best action in accordance with some implementations is shown. The method 808 begins by receiving 1102 prescription information, therapeutic alternatives, and problem information. For example, the prescription information may include prescription and related information provided by the prescription intake module 142 to the next best action module 150. This information may include the prescription, the prescriber, the patient, the price of the medication, etc. The therapeutic alternatives may be received by the next best action module 150 from the therapeutic alternative recommendation module 146. The problem information is provided by the problem identification module 144 and sent to the next best action module 150. Next, the method 808 determines 1104 possible actions in response to the problems that were received in block 1102. This process has been described above with reference to the action determination module 702. The method 808 continues by determining and excluding 1106 certain actions from consideration. As noted above, certain actions may be excluded and their exclusion is performed by the action filter module 706. Block 1106 is depicted with dashes in FIG. 11 indicating that the exclusion step is optional. For example, some actions may be excluded because they do not fully resolve the problem. Other actions may be excluded because the optimization process cannot be performed on them. Next, the method 808 prioritizes 1108 actions based on one or more criteria. Typically, the method 808 prioritizes the actions based upon how much impact they will have in optimizing or concluding the prescription fulfillment process. In one example, particular actions with many dependencies in the workflow are prioritized and ranked higher than other actions which can be performed at any time. Examples of prioritization have been described above with regard to the operation of the action ranking module 704 and the rules logic 708 of the next best action module 150. Once the actions have been prioritized in block 1108, the method 808 continues by selecting 1110 one of the actions as the next best action and sending that next best action back to the action execution module 152.

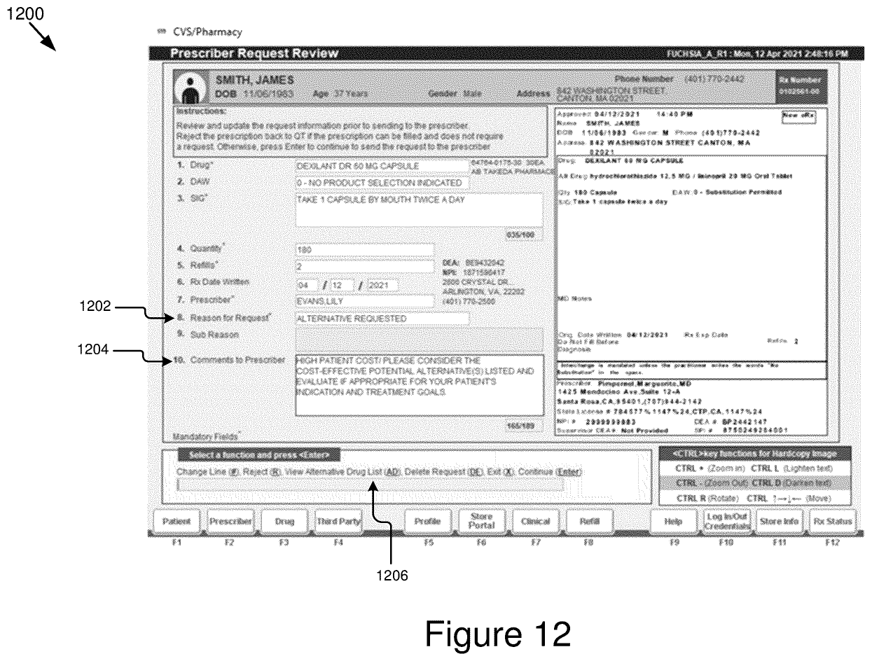

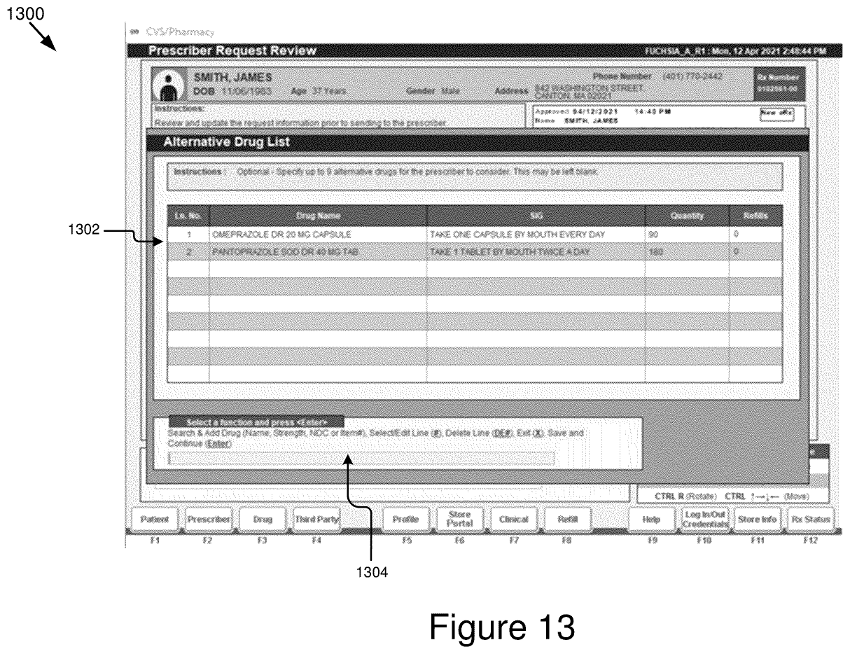

FIGS. 12 and 13 are graphic representations of example user interfaces for a pharmacist to interact with the pharmacy system 100 in accordance with some implementations.

FIG. 12 illustrates an example user interface 1200 presented to a pharmacist or pharmacist technician to be used in the process of fulfilling a prescription. The user interface 1200 is generated by the prescription optimization system 140 to allow the pharmacist to efficiently interact with and send messages/requests to a prescriber in the event any problems are encountered. The prescription optimization system 140 advantageously pre-populates many of the fields in the user interface 1200 based on patient information, prescription information and therapeutic alternatives. In some implementations, the user interface 1200 and the data for it are generated by the next best action module 150. The user interface 1200 presents various information about the patient such as their name, address, age etc. The user interface 1200 may also present information about the drug, a copy of the prescription, and other prescription information. The prescription optimization system 140 is particularly advantageous because it pre-populates the reason for the request 1202 to the prescriber, which in this example is a request for approval of a therapeutic alternative. Additionally, the prescription optimization system 140 may also pre-populate an explanation field 1204 labeled “comments to the prescriber” with an explanation as to why the pharmacy is asking for approval for a therapeutic alternative. This interface 1200 is particularly advantageous because it improves the efficiency of the pharmacist because the fields are auto-populated and the pharmacist need only review the fields for their clinical appropriateness and approve them to generate a request to the prescriber. The user interface 1200 also includes a function box including the ability for the pharmacist to view the list of alternative drugs 1206 as will be described in more detail below with reference to FIG. 13.

FIG. 13 illustrates an example user interface 1300 for a pharmacist to interact with the pharmacy system 100. In particular, the user interface 1300 is shown after the pharmacist has selected view the list of alternative drugs 1206 function in the user interface 1200 described above with reference to FIG. 12. Once the pharmacist selects the view alternative drug list function 1206, the prescription optimization system 140 transitions from the user interface 1200 of FIG. 12 to the user interface 1300 of FIG. 13. As can be seen, the user interface 1300 includes a list 1302 of therapeutic alternatives that are being identified and sent in the request to the prescriber as possible therapeutic alternatives. Again, the prescription optimization system 140 is particularly advantageous because this information is automatically retrieved, determined, and pre-populated so that it may be part of the request to the prescriber. This eliminates any manual entry by the pharmacist. As shown in the function box 1304, this user interface 1300 also allows the pharmacist to delete any therapeutic alternative listed or add any therapeutic alternatives not shown based on the pharmacist's determination of what is accurate and clinically appropriate for the patient. As can be seen, the user interface 1300 also allows the pharmacist to access the therapeutic alternative database 130 to look up therapeutic alternatives.

Referring now to FIGS. 14-17 example user interfaces generated by the prescription optimization system 140 will be described. These example user interfaces show interfaces and information that are generated by the prescription optimization system 140 cooperating with the pharmacy computing device 108. The below interfaces will now be described with reference to interfaces used to interact with the patient and presented on their patient computing device 120, in particular, a mobile phone in this example. It should be understood that the information generated and presented by the prescription optimization system 140 may be presented in similar user interfaces for a desktop computer, a laptop, a tablet, or workstation in other implementations.

FIG. 14 illustrates interfaces 1402, 1404 and 1406 generated by the prescription optimization system 140 for presentation of an action to the patient, and solicitation of a decision to address a problem identified by the prescription optimization system 140. The prescription optimization system 140 generates and presents or causes to be displayed a first user interface 1402 referred to as an action notes page that provides an indication that action is needed by the patient. This user interface 1402 may also include information about the particular prescription, the details of the problem, and pricing information. The prescription optimization system 140 next presents a second user interface 1404 referred to as a detail and options page that presents an indication of the problem and a series of radio buttons with different choices that the user may select. In the specific example illustrated in FIG. 14, the problem is that the order prescription is out of stock. Once the user selects an option and submits their choice, the prescription optimization system 140 generates the success page confirming to the patient that their choice has been received by the prescription optimization system 140.

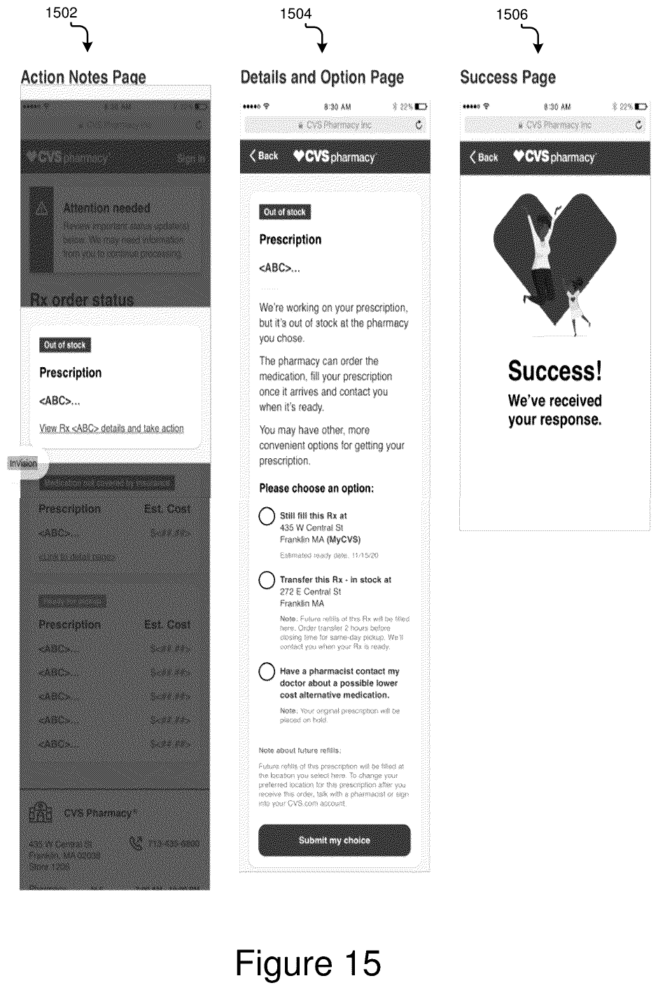

FIG. 15 illustrates interfaces 1502, 1504 1506 generated by the prescription optimization system 140 for presentation of different options that may be presented to the user based on a similar problem. The user interfaces in both FIGS. 14 and 15 both relate to an out-of-stock problem. However, the options for resolving the same problem are different. FIGS. 14 and 15 illustrate that additional user interfaces beyond those presented in FIGS. 14-17 can be used based on different possible alternatives to resolve a particular problem. As shown in FIG. 15, the user interfaces and interaction generated by the prescription optimization system 140 again include actions note page 1502, a detailed in option page 1504, and a success page 1506. However, in this example the options presented in the user interface 1504 are different and include a choice to fill the prescription at the original pharmacy where the prescription was submitted, transfer the prescription to a pharmacy at a different location, or have the pharmacist contact the prescriber about a different lower cost alternative. Based on the differences between FIGS. 14 and 15, it can be understood how the user interfaces generated by the prescription optimization system 140 may be varied based upon different factors including efficiently resolving the problem, providing the patient with information to make a decision, presenting options most likely to be selected by the patient, and various other factors described above for the system 100 components.

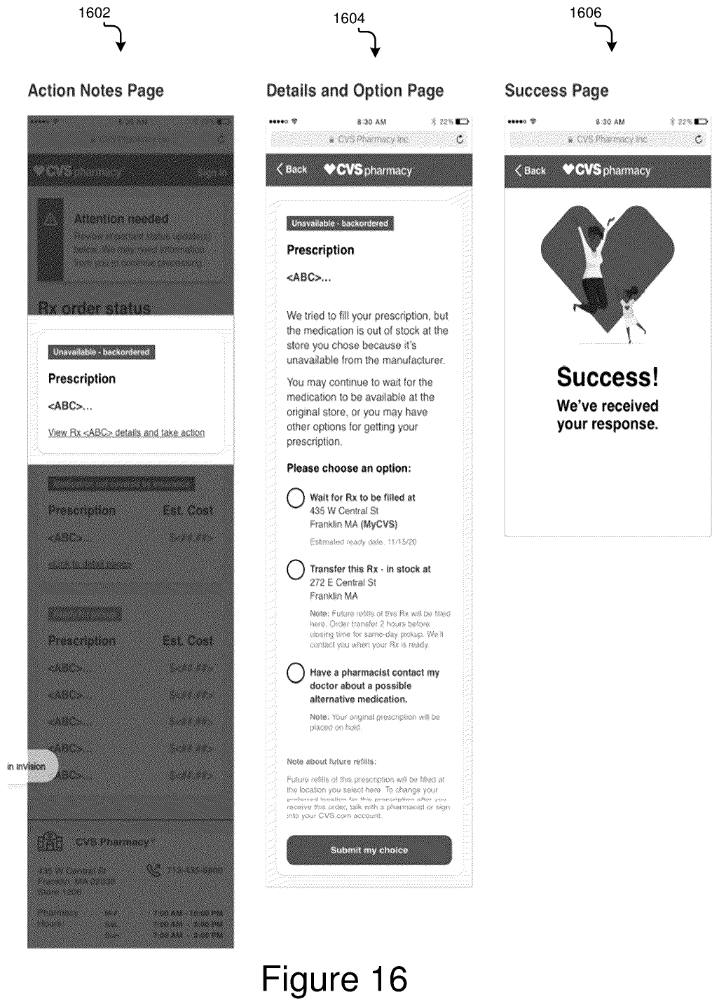

FIG. 16 illustrates interfaces 1602, 1604 and 1606 generated by the prescription optimization system 140 for presentation and resolution of the problem. In this specific example, the problem is that the prescription is on backorder. Again, the prescription optimization system 140 generates a user interface 1602 for presenting the required action and the prescription in the action notes page, a user interface 1604 for providing the details about the problem and options for resolving it in the details and options page, and a user interface 1606 for confirming to the patient that their selection has been accepted in a success page.

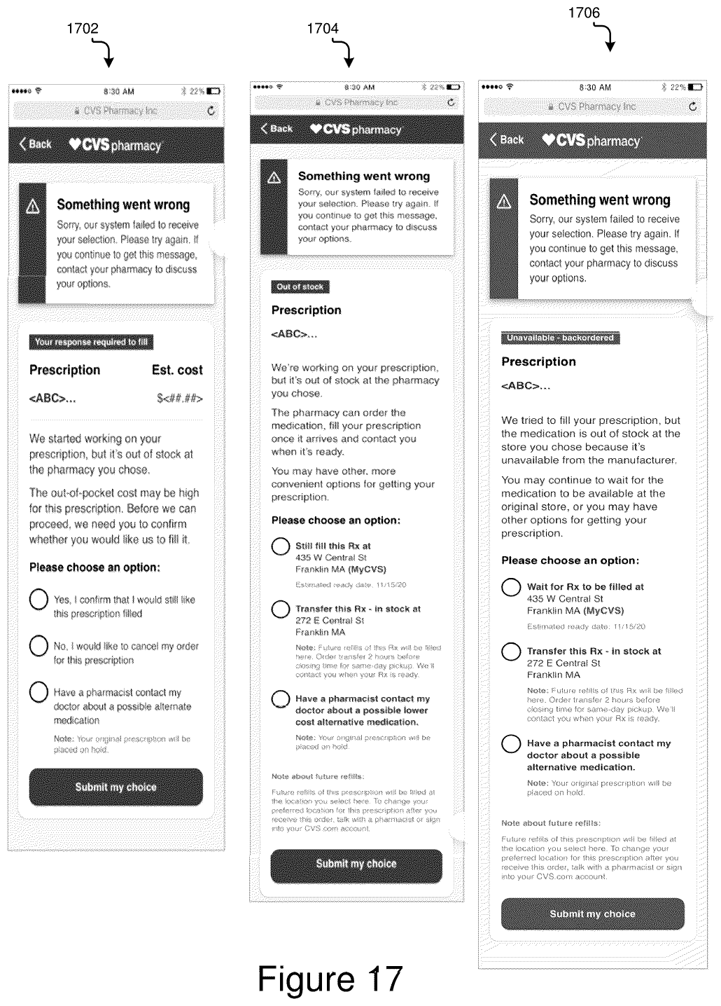

Referring now to FIG. 17, user interfaces 1702, 1704, and 1706, generated by the prescription optimization system 140 for indicating that there has been an error in communicating between the pharmacy optimization system 140 and the patient computing device 120 are shown. The first user interface 1702 illustrates one example of an error in which the prescription optimization system 140 does not receive a reply from the patient for any reason. In the user interface flow described above with reference to FIG. 14, instead of ending with a user interface success page 1406, the user interface 1702 indicating an error is displayed. The user interface 1702 includes an indication that there was an error, and re-presentation of the options to resolve the problem that were previously presented. Similarly, user interface 1704 shows an error message indicating that a submission in the user interface flow of FIG. 15 was not received. This error user interface 1704 again indicates an indication that there was an error, and re-presentation of the options for resolving the out-of-stock problem and is displayed rather than user interface 1506. The third error user interface 1706 illustrates the user interface if an error occurred during the submission of response to the user interface flow of FIG. 16.

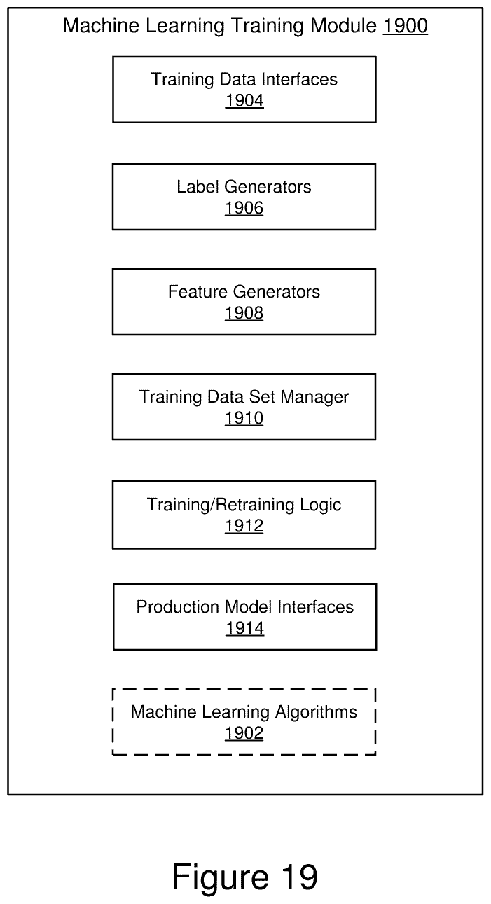

Referring now to FIG. 18, a block diagram illustrating an example of an insurance coverage module 404 in accordance with some implementations is shown. In some implementations, insurance coverage module 404 may operate as a component of problem identification module 144 and interface with next best action module 150 and/or therapeutic alternative recommendation module 146. In some implementations, the insurance coverage module 404 may operate as a separate component that incorporates features similar to problem identification module 144, next best action module 150, and/or therapeutic alternative recommendation module 146. Insurance coverage module 404 may generate next best actions to proactively and/or responsively address problems with insurance claim submissions to insurance adjudication systems in order to automatically prevent or resolve rejections by those systems. In some implementations, insurance coverage module 404 may operate on parameter sets derived from prescription information to generate and initiate one or more next best action service calls, such as to action execution module 152, to support successful submission or resubmission of an insurance claim for that prescription and patient. Insurance coverage module 404 may be comprised of control logic that leverages a series of rule sets, machine learning models, and component interfaces to generate next best action service calls for a specific set of next best action services available for insurance problem resolution. The control logic may include pre-model rules module 1802, one or more special resolution machine learning models 1804.1-1804.n, a general resolution machine learning module 1806, and a post-model rules module 1808. The control logic may optionally be supported by a rules engine 1800. In some implementations, rules engine 1800 may be a Boolean logic engine configured to automatically read a rules set and corresponding input parameters to make complex logical rules decisions in volume processing. For example, rules engine 1800 may process rule sets corresponding to pre-model rules module 1802 and post-model rules 1806 to evaluate inclusion or exclusion conditions for one or more machine learning models and/or next best actions. In some implementations, the control logic may include an adjudication interface 1810 for initiating a submission of an insurance claim in response to completion of one or more next best actions. Additionally, adjudication interface 1810 may receive adjudication decisions, such as rejection notifications including one or more rejection codes, to trigger processing or further processing by the control logic. For example, adjudication interface 1810 may include a network messaging interface or application protocol interface (API) for calling claim application 116 or a third-party insurance adjudication system. In some implementations, the machine learning training module 1900 may be included in or interface with insurance coverage module 404 for training and/or retraining one or more machine learning models, as described with regard to FIG. 19. Selected actions may be sent by a corresponding next best action service call to the pharmacy computing device 108, in particular the action execution module 152, to perform the selected action.

In the example shown, insurance coverage module 404 includes a therapeutic alternatives interface 1820 to initiate one or more service calls for changing the prescribed drug, such as calling therapeutic alternative recommendation module 146. Insurance coverage module 404 further includes a quantity change service 1822, an insurance plan change service 1824, a cash pay change service 1826, a future fill date change service 1828, a prescriber request service 1830, a prior authorization request service 1832, and a customer service prompt service 1834. The control logic may be configured to generate a next best action service call directed to one of these services, as further described below.

Pre-model rules module 1802 may be steps, processes, functionalities, software executable by a processor, or a device including routines for evaluating a set of rules for excluding prescriptions from processing through one or more machine learning models. For example, pre-model rules module 1802 may receive a parameter set corresponding to selected prescription information and invoke rules engine 1800 to evaluate the parameter set against a pre-model set of rules. These rules may include direct, logical correlations between one or more parameter values from the parameter set that determine or exclude specific next best actions without being processed through the machine learning models. The pre-model rule set may capture scenarios which are able to be clearly laid out with business logic to automatically apply a next best action without the application of machine learning or are required to be treated differently for regulatory purposes. For example, pre-model rules that may be applied to the input parameter set may include:
1. Any rejection associated to a workman's compensation plan is not eligible for any next best action and is sent back to the store to be manually addressed
2. Any rejection which meets the below criteria is switched to being paid in cash:
   a. For an over-the-counter (OTC) drug
   b. Costs less than a fixed dollar amount
   c. Has specific third-party rejection codes
   d. Is rejected by a commercial plan
3. Any rejection which meets the below criteria is scheduled to be filled on a future date:
   a. Not a coordination of benefits scenario
   b. Has free text messaging which does not contain disallowed phrases
   c. Has free text messaging which does have a date in the future
   d. Has specific third-party rejection codes
4. Any rejection which meets the below criteria has a prescriber request sent to change the therapy:
   a. Not a coordination of benefits scenario
   b. Not a controlled substance
   c. Is not eligible to be returned to stock
   d. Is for an allowed class of therapies
   e. Has specific third-party rejection codes
   f. Has third party provided preferred alternatives
   g. Costs more than a fixed dollar amount
   h. Where the patient does not have a history of using alternative payment methods
5. Any rejection which meets the below criteria is edited to bill the primary insurance only:
   a. Is a coordination of benefits scenario
   b. Secondary payer returns specific third-party rejection codes
   c. Has free text messaging which does not contain dis-allowed phrases
6. Any rejection which meets the below criteria is changed to bill for the brand name alternative drug instead of the generic:
   a. Not a coordination of benefits scenario
   b. Is not eligible to be returned to stock
   c. Is for an allowed class of therapies
   d. Does not have a dispense as written (DAW) code of 1
   e. Has specific third-party rejection codes
   f. Contains a DAW code of 6, 7 or 9 as part of its free text messaging
   g. Has third party provided preferred alternatives which are from the same therapeutic class but are brand name
      The pre-model rule set may map a set of parameter values to conditions for specific next best actions or conditions for excluding further processing of the prescription by insurance coverage module 404. Prescriptions with parameter sets meeting a condition in the rule set may initiate the next best action service call specified by that rule or, in the case of exclusions, be returned with no next best action or further resolution by insurance coverage module 404. In exclusion cases, pre-model rules module 1802 may return a reason value corresponding to the rule condition that was met for the exclusion and that reason value may be passed to other system components and/or displayed through the pharmacy computing system.

In some configurations, the pre-model rule set may interact with special resolution models 1804 in a more complex sequence that interleaves rule-based decision-making with the various special resolution models in a sequence that precedes processing remainder cases through general resolution model 1806. For example, the pre-model rule set may be divided into a series of subsets, where a first subset is completed prior to a first special resolution model, then a second subset is completed prior to a second special resolution model, and so on until all of the rules in the rule set and special resolution models have been evaluated and any remaining cases are sent to the general resolution model. In some configurations, the interleaved rules and special resolution models may be organized linearly based on priority or likelihood of success for more efficient processing of the sequence. In some configurations, the interleaved rules and special resolution models may be mapped to a decision-tree structure with decisions based on parameter values and/or outputs of prior rule or model evaluations.

Special resolution models 1804 may be steps, processes, functionalities, software executable by a processor, or a device including routines for processing a parameter set through an inference engine trained by machine learning for identifying and handling a specific resolution case. For example, a machine learning model similar to machine learning model 412 or machine learning model 710 may be trained based on training data labeled for specific rejection codes and corresponding next best actions to provide higher confidence next best action recommendations than general resolution machine learning model 1806. Special resolution models 1804 may include parameter changes as part of the next best action generation and may include specially trained inference engines for generating the change values for those parameter changes. In some implementations, special resolution models 1804 may each be specialized models trained for a highly specific rejection type that is a combination of rejection code and parameter values corresponding to a specific next best action and parameter changes to make that next best action successful for resolving an insurance claim problem. These models may be targeted to predict specific actions to resolve these rejections and can incorporate an ensemble model structure, with initial models used to identify the applicability of an action and subsequent models to predict specific parameter value changes associated to the action. For example, for a specific set of rejection codes, an initial model may be trained to determine if the quantity and days of a prescription needs to be changed to achieve a paid claim. If this first model flags this rejection as needing a changed quantity/days supply, then a subsequent model is used to predict the specific quantity and days supply that will receive a paid claim. As another example, for another specific set of rejection codes and rejected drug types, an initial model will determine if a prescription needs a prior authorization (PA) from the patient's prescriber to be paid. If a PA is required, a subsequent model may be used to predict the likelihood that the patient's prescriber will submit this prior authorization. Based on this likelihood, the next best action service call may be to initiate the PA, attempt to change the drug, or take some other non-automated action, such as initiating a pharmacist or other customer service staff to contact the patient. Each special resolution model 1804 may produce a confidence value for their prediction of the problem, next best action, and/or parameter change value and the control logic may use threshold comparisons and/or ranking of confidence values from multiple special resolution models to determine which special resolution models are used for a particular prescription resolution.

General resolution model 1806 may be steps, processes, functionalities, software executable by a processor, or a device including routines for processing a parameter set through an inference engine trained by machine learning for determining which next best action of a set of next best actions is most likely to resolve an insurance problem. For example, a machine learning model similar to machine learning model 412 or machine learning model 710 may be trained based on training data labeled for a variety of rejection codes and corresponding next best actions to select a highest confidence next best action recommendation for the cases that have not been handled by pre-model rules module 1802 or special resolution models 1804. General resolution model 1806 may include a general-purpose machine learning model trained to predict which next best action, out of a defined set of next best action service calls available, is most likely to resolve the observed rejection. This model may use a large parameter set based on a range of information provided by the internal pharmacy systems and from the third party who is rejecting the prescription, particularly a set of rejection codes for the prescriptions. In some parameter sets, this information may be combined with patient and therapy-level historical information that has been identified as having significant predictive power. For example, general resolution model 1806 may use a multi-class classifier model to process features from a large parameter set to generate confidence value outputs for each possible next best action in the set of actions.

Post-model rules module 1808 may be steps, processes, functionalities, software executable by a processor, or a device including routines for evaluating a set of rules for excluding outcomes from one or more machine learning models that may violate known internal, third party, and/or regulatory conditions. For example, post-model rules module 1808 may receive a parameter set corresponding to selected prescription information and invoke rules engine 1800 to evaluate the parameter set against a post-model set of rules. These rules may include direct, logical correlations between one or more parameter values from the parameter set that exclude specific next best actions or parameter values therein that may be output by the various machine learning inference engines. The post model rules may edit the machine learning output to ensure that they comply with relevant regulations and provide clinically valid outputs. For example, post model rules that may be applied to the output of general resolution model may correlate to specific next best action selections:
1. Next Best Action (NBA) 1: Disallowed words in free text messages, no alterative quantity/drug substance or no alternative national drug code (NDCs) may be excluded from some therapeutic alternative next best actions.
2. NBA 2: Any coordination of benefits, certain therapeutic classes or no alternative NDCs may be excluded from some therapeutic alternative next best actions.
3. NBA 3: Controlled substances, have already sent this NBA, wrong dosage forms, and certain therapeutic classes or daily dosing changes may be excluded from quantity change actions.
4. NBA 4: Controlled substances, any coordination of benefits, disallowed words in free text messages, or condor code not Commercial or Med D or in disallowed list or no unbilled third-party premium collection TPPCs may be excluded from insurance plan change actions.
5. NBA 5: Controlled substances, any coordination of benefits, disallowed words in free text messages, condor code not Commercial or Med D or in disallowed list, or have history of using other payment methods may exclude cash pay change actions.
6. NBA 7: Controlled substances, cost below set threshold, certain therapeutic classes, or do not have alternative drugs may exclude prescriber request actions,
7. NBA 8: Controlled substances, cost below set threshold, OTC drugs, or certain therapeutic classes may exclude prior authorization request actions.
   The post-model rule set may map a set of parameter values to conditions for excluding specific next best actions and validating the output of the machine learning models. Prescriptions with parameter sets meeting a condition in the rule set may cause the prescription to be returned with no next best action or further resolution by insurance coverage module 404. Post-model rules module 1808 may return a reason value corresponding to the rule condition that was met for the exclusion and that reason value may be passed to other system components and/or displayed through the pharmacy computing system.

Insurance coverage module 404 may include interfaces and services for initiating next best actions determined by the control logic. Each interface or service may include steps, processes, functionalities, software executable by a processor, or a device including routines for initiating a next best action in the set of next best actions identified for resolving insurance adjudication problems. Therapeutic alternative interface 1820 may include a messaging or API interface for calling therapeutic alternative recommendation module 146 to determine and change the drug in the prescription to a therapeutic alternative. For example, one next best action service may be called to change the NDC value to a therapeutically equivalent indicated by the third-party insurer or based on a lookup table of historically paid patterns. In another example, a next best action service may be called to change the NDC value from a brand name to a generic equivalent or vice versa based on coverage by the particular insurance plan. Quantity change service 1822 may include next best action calls that determine a changed quantity or days supply value for modifying the prescription information. For example, a next best action service may be called to determine a reduced quantity or number of days of supply of the drug that complies with what the patient's insurance has historically paid out. Insurance plan change service 1824 may include next best action calls that determine a change in the insurance carrier or plan to which the prescription should be submitted. For example, a next best action service may be called to determine that there is one or more other insurance plans associated with a patient's profile and, if so, which is most likely to pay for the particular prescription. Cash pay change service 1829 may include next best action calls that determine that the payment type should be changed to cash. For example, a next best action service may be called to determine that no insurance plan covers the prescription and the patient should pay directly for the prescription at pickup. Future fill date change service 1828 may include next best action calls that determine a future date for filling the prescription. For example, a next best action service may be called to determine that the prescription would be payable if filled at a future date, such as based on refill schedule, change in coverage, or other predicted time-based event. Prescriber request service 1830 may include next best action calls that generate and send a request to the prescriber to change the NDC value and/or quantity to an alternative. For example, a next best action service may be called to initiate a prescriber request message populated with one or more therapeutic alternatives and/or quantities covered by the patient's insurance. In some configurations, the list of therapeutic alternatives may be generated by a next best action service call through therapeutic alternative interface 1820. Prior authorization request service 1832 may include next best action calls that generate and send prior authorization requests to the patient's insurer. For example, a next best action service may be called to initiate a preauthorization request to authorize the therapy in the prescription, where preauthorization is required by the insurance plan. Customer service prompt service 1834 may include next best action calls that generate an informational prompt to one or more customer service systems or representatives. For example, a next best action service may be called to initiate an informational prompt to the pharmacy staff at a retail pharmacy processing the prescription to initiate communication or outreach to the patient regarding the prescription, such as missing information related to insurance coverage or determination that the pharmacy does not participate in the patient's insurance plan. This set of next best actions is exemplary and should not be considered exhaustive or limiting to the different next best actions services that could be included in insurance coverage module 404.

Referring now to FIG. 19, a block diagram illustrating an example of machine learning training module 1900 in accordance with some implementations is shown. In some implementations, one or more machine learning models in insurance coverage module 404 and/or other components of prescription optimization system 140 may by trained by machine learning training module 1900. For example, general resolution model 1806 and each special resolution model 1804.1-1804.n may be trained and/or retrained by machine learning training module 1900 prior to production deployment. Machine learning training module 1900 may optionally support any number of machine learning algorithms 1902 and corresponding topologies and training methods. Machine learning algorithms 1902 may include geometric systems like nearest neighbors and support vector machines, probabilistic systems, evolutionary systems like genetic algorithms, decision trees, neural networks, associated with decision trees, Bayesian inference, random forests, boosting, logistic regression, faceted navigation, query refinement, query expansion, singular value decomposition and the like. Machine learning training module 1900 may use supervised learning, semi-supervised learning, or unsupervised learning for building and training the machine learning models based on the type of data available and the particular machine learning technology used for implementation. Machine learning training module 1900 may include one or more training data interfaces 1904, label generators 1906, feature generators 1908, training data set manager 1910, training/retraining logic 1912, and production model interfaces 1914.

Training data interfaces 1904 may be steps, processes, functionalities, software executable by a processor, or a device including routines for gathering parameter data sets from a plurality of data sources for use in training the coefficients of machine learning algorithms 1902 for specific machine learning models. For example, training data interfaces 1904 may interface with one or more data storage systems in an enterprise pharmacy data warehouse, such as the production data supporting pharmacy operations application 110, POS application 112, prescription intake module 142, action execution module 152, claim application 116, formulary information 118. These data stores may include a variety of prescription information in structured data repositories that may be mined and parameterized to populate defined parameter sets to support machine learning training. For example, data values from prescription records and associated patient records, insurance carrier/plan records, adjudication records, prescriber records, pharmacy records, transaction records, and/or historical data related to each data type. In some implementations, training data sets may be curated from data sets that are selected from and/or based on historical production data, but have been cleansed, labeled, and validated for specific model training purposes. For example, the training data set for one or more models may be based on selected data records which have been directly logged via specific production systems that remove dependencies on retail pharmacy systems, historical patient/drug information, or records of third party rejection information that may have different or inconsistent levels of data retention and data accuracy which can adversely impact reliable training data.

Label generators 1906 may be steps, processes, functionalities, software executable by a processor, or a device including routines for labelling instances of input parameter sets in the training data set for known outcomes (e.g., ideal problem identification or next best action selection) that may be used in training the coefficients of machine learning algorithms 1902 for specific machine learning models. For example, label generators 1906 may allow the instances of each parameter set in a training data set to be mapped to the desired output for that instance to support training and/or retraining of a machine learning model. Label generators 1906 may be configured to generate training data sets that accurately represent the production environments. In some implementations, label generators 1906 may operate on production data received and processed through insurance coverage module 404 to refine and improve models over time through training new models and/or retraining prior models. For example, adjudication records may be stored by insurance coverage module 404 in an adjudication record data store, such as a relational database, and logic used to generate machine learning labels from the prescription information and adjudication records may use similar logical rules to those used to monitor model performance and validate model output, such as pre-model and post-model rules. In some implementations, the labels used may each correspond to the desired next best action and the set of labels used for any given model may correspond to the subset of next best actions handled by that model. For example, the general resolution model may be trained with data labeled with the set of seven possible next best actions that may be generated, while special resolution models may be trained with data labeled and filtered for a subset of next best actions.

Feature generators 1908 may be steps, processes, functionalities, software executable by a processor, or a device including routines for determining feature sets from parameters in the training data sets for use in training the coefficients of machine learning algorithms 1902 for specific machine learning models. For example, feature generators 1908 may apply feature transformations to the parameter values in the parameter sets, which may include combinations of structured and free text data, to determine a set of feature vectors for processing through machine learning algorithms 1902 using training/retraining logic 1912. In some implementations, a number of feature engineering rules may be applied to the training data set to generate the feature set input to the models for training. For example, where numeric features are used, missing values may be imputed as zeros and/or categories may be frequency encoded using their frequency in the training data set. As another example, text may be encoded using an overlap metric that captures a most common word set for each label (e.g., top 50 most common words), calculates a percentage of words in text which are contained in most common word lists for each label in the set of labels being used, and reduces overfitting by doing the most common word generation on a data set which is independent from the actual training data sets used.

Training data set manager 1910 may be steps, processes, functionalities, software executable by a processor, or a device including routines for managing parameter data sets from a plurality of data sources for use in training the coefficients of machine learning algorithms 1902 for specific machine learning models. For example, training data set manager 1910 may use training data interfaces 1904 to gather raw data for training data generation, select specific parameter sets for training specific machine learning models, and process it through label generators 1906 and feature generators 1908 for training machine learning models. In some implementations, training data set manager 1910 may be used to establish training data sets for each machine learning model and integrate additional data into that training data set across operating times to support retraining by training/retraining logic 1912. In some configurations, training data sets may be selected and validated to balance the set or subset of labels represented in the training data set.

Training/retraining logic 1912 may be steps, processes, functionalities, software executable by a processor, or a device including routines for initiating and managing the training of the coefficients of machine learning algorithms 1902 for specific machine learning models. For example, training/retraining logic 1912 may include control logic for using training data interfaces 1904, label generators 1906, feature generators 1908, and training data set manager 1910 to support training machine learning models and placing them into production use using production model interfaces 1914. In some implementations, training retraining logic 1912 may include a combination of machine learning training automation and manual selection and validation of training data sets, labels, features, and machine learning algorithms for desired machine learning model performance. Over time, specific models with validated performance in production use may be further automated for routine monitoring, validation, and retraining. For example, conditions may be defined within the system for evaluating model performance and periodically triggering retraining based on updated training data sets from production operation of insurance coverage module 404.

Production model interfaces 1914 may be steps, processes, functionalities, software executable by a processor, or a device including routines for moving specific trained machine learning models and corresponding model algorithm, topology, and coefficients from machine learning training module 1900 to corresponding production systems, such as insurance coverage module 404 and/or other components of prescription optimization system 140 using machine learning models. For example, production model interfaces 1914 may migrate a trained or retrained instance of one or more machine learning models to the corresponding production systems for processing user prescriptions for optimization. In some implementations, production model interfaces 1914 may include a messaging interface, direct memory access, and/or an API for storing or updating a production instance of a machine learning model in another system component.

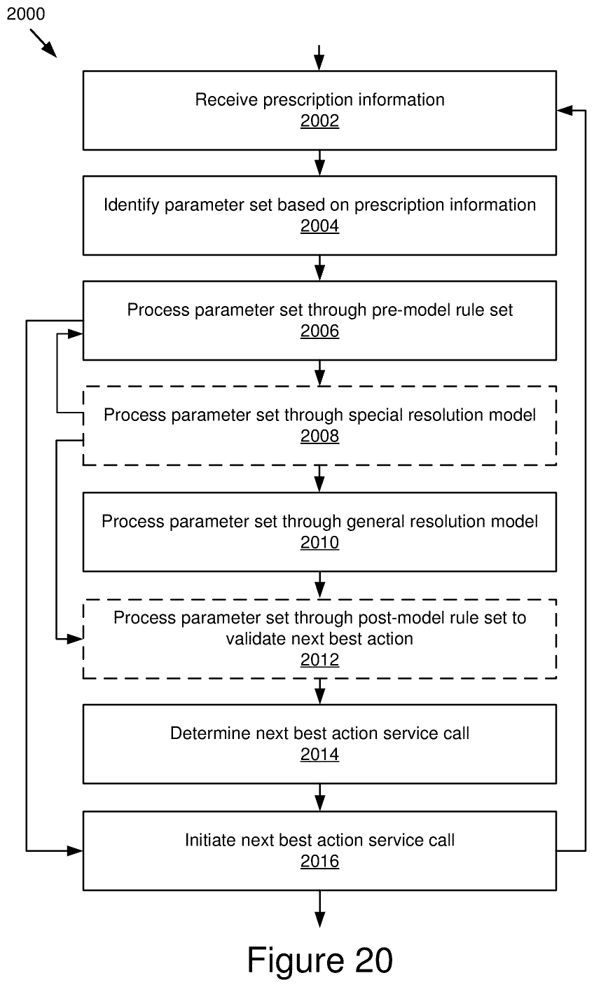

Referring now to FIG. 20, a method 2000 for optimizing fulfillment of a prescription in accordance with some implementations is described. At step 2002, prescription information may be received. For example, a prescription optimization system may receive a prescription for a patient that is in the process of being filled from a pharmacy computing system, user device (patient or prescriber), and/or other prescription request system. At step 2004, a parameter set may be identified based on the prescription information. For example, the prescription optimization system may select parameter values associated with the prescription record and related records in or accessible from the prescription optimization system. At step 2006, the parameter set may be processed through a pre-model rule set. For example, the prescription optimization system may include a rules engine or rules logic that applies a set of logical rules to determine whether the parameter set corresponds to conditions for a specific next best action and/or is excluded from further processing through one or more machine learning modes for determining next best actions. Parameter sets meeting the conditions of one or more rules in the rule set may cause method 2000 to proceed to step 2016 and skip processing at steps 2008-2014. At step 2008, the parameter set may optionally be processed through one or more special resolution models. For example, the prescription optimization system may include one or more special resolution machine learning models trained to handle a specific case that determines a specific next best action service to be called. Parameter sets meeting a confidence threshold determination of a next best action from a special resolution model may cause method 2000 to proceed to step 2012 and skip processing at step 2010. In some configurations, the pre-model rule set may include multiple subsets or stages that are interleaved with one or more special resolution models such that special resolution models may selectively return method 2000 to step 2006 for processing of further rule subsets when a next best action is not successfully determined by that special resolution model. At step 2010, the parameter set may be processed through a general resolution machine learning model. For example, the prescription optimization system may include a general resolution model for a larger set of next best action services (and corresponding labels in the machine learning model) and return next best action confidence values for a plurality of next best action services. At step 2012, the parameter set may be processed through a post-model rule set to validate the next best action and any parameter change values from the preceding models. For example, the prescription optimization system may use the rules engine or rules logic to apply another set of logical rules to determine whether the decision outputs from the machine learning models correspond to conditions for invalidating the next best action identified by the models. At step 2014, a next best action service call may be determined. For example, the prescription optimization system may use the model outputs validated at step 2012 to select the next best action service call and populate it with appropriate parameter values for automatically initiating and completing the action. At step 2016, a next best action service call may be initiated. For example, the prescription optimization system may initiate a next best action service call to another system component with the needed parameters for that action in order to automate completion of that next best action. In some implementations, method 2000 may return to step 2002 with updated prescription information following completion of the next best action service call to determine a next optimization (next best action) if the prescription is still pending.

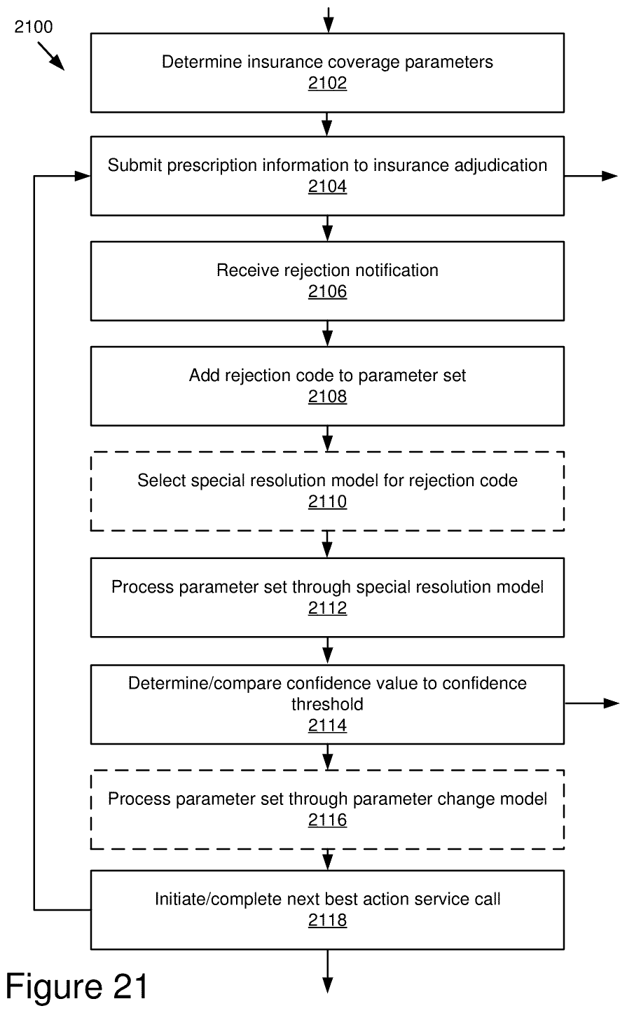

Referring now to FIG. 21, a method 2100 for determining a next best action following a rejection from an insurance adjudication system in accordance with some implementations is described. Method 2100 may operate in conjunction with one or more steps of method 2000. At step 2102, insurance coverage parameters may be determined. For example, an insurance coverage module may be configured to select a parameter set related to the submission of insurance claims to the insurance adjudication system of an insurance provider. At step 2104, prescription information may be submitted to insurance adjudication. For example, the insurance coverage module may interface with an insurance claim application for submitting insurance claims to the insurance adjudication system for the insurance carrier associated with a prescription claim. If the insurance claim is accepted, method 2100 may be concluded. Otherwise, a rejection may be received at step 2106. At step 2106, a rejection notification may be received. For example, the insurance coverage module may receive, directly or indirectly, a notification message indicating one or more rejection codes for rejecting the previously submitted insurance claim for the prescription. At step 2108, rejection codes may be added to the parameter set. For example, the insurance coverage module may add one or more rejection codes from the rejection notification to the parameter set being processed for next best actions. At step 2110, a special resolution model may optionally be selected based on the rejection code. For example, the insurance coverage module may include a plurality of special resolution machine learning models and may be indexed by rejection codes to determine which special resolution model is initiated and/or an order in which the special resolution models are processed. In some implementations, a pre-model rule set may be processed prior to step 2110 as described for step 2006 in method 2000. At step 2112, the parameter set may be processed through one or more special resolution models. For example, the insurance coverage module may include one or more special resolution models for handling insurance rejections corresponding to specific next best actions and may process a selected special resolution model from step 2110 or sequentially process all special resolution models until a confidence threshold is met or not met at step 2114. At step 2114, confidence values from the special resolution models may be determined and compared to one or more confidence thresholds. For example, the insurance coverage module may determine one or more confidence values from each special resolution model processed at step 2112 and compare them to one or more confidence thresholds for determining a next best action from the special resolution models. If a threshold is met, method 2100 may proceed to step 2116 and/or 2118. If no confidence threshold is met, method 2100 may end and operation may proceed to step 2010 of method 2000 for processing through a general resolution model. At block 2116, the parameter set may be optionally processed through a parameter change model associated with a special resolution model. For example, the insurance coverage module may use an ensemble configuration of multiple special resolution models to separately determine a confidence value for next best action selection and one or more parameter change values to support initiation/execution of the selected next best action. At block 2118, a next best action service call may be initiated and completed. For example, the insurance coverage module may proceed similarly to steps 2012-2016 in method 2000 to initiate a next best action based on the machine learning model and rule processing. In response to the next best action service completing the called service and updating the prescription information, method 2100 may automatically return to step 2104 to submit the updated prescription and insurance coverage parameters to the insurance adjudication system in an attempt to resolve the prior rejection.

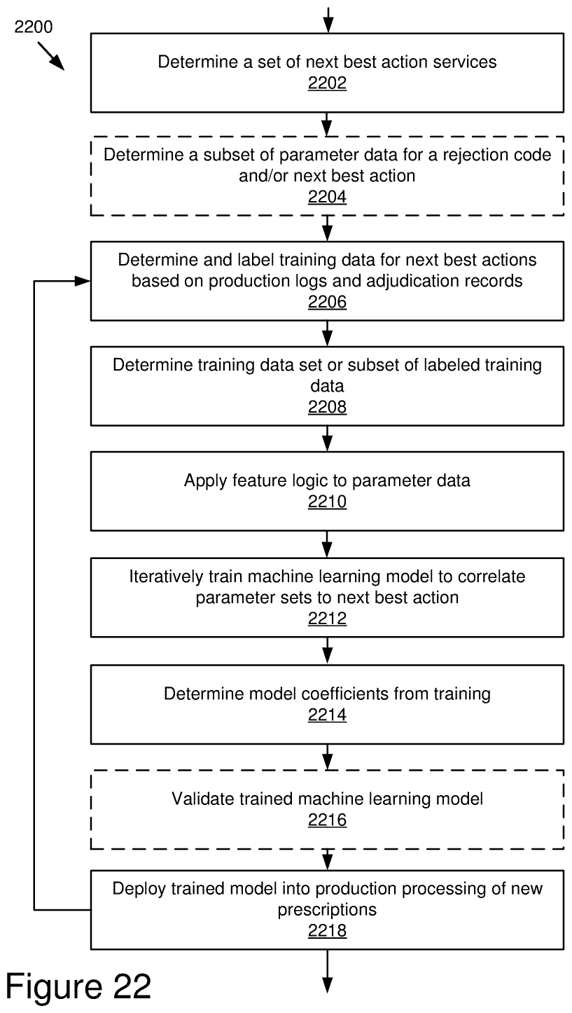

Referring now to FIG. 22, a method 2200 for training machine learning models for determining next best actions in accordance with some implementations is described. At step 2202, a set of next best action services may be determined. For example, a machine learning training module may determine a set of next best actions for resolving an insurance claim rejection. At step 2204, a subset of parameter data may be optionally determined for a rejection code and/or next best action. For example, in some implementations, the machine learning training module may determine one or more special resolution models that will be based on one or more specific rejection codes and one or more next best actions for resolving the rejection codes and a corresponding subset of parameter data may be selected for the training data. At step 2206, training data may be determined and labeled for the next best actions based on production logs and adjudication records. For example, the machine learning training module may select and process parameter data sets and corresponding production logs and adjudication records to determine corresponding next best action labels. At step 2208, the training data set or subset of labeled training data is determined. For example, the machine learning training module may iteratively process parameter sets to generate labels until a training data set with the desired distribution of parameter sets and labels is achieved. At block 2210, feature logic may be applied to the parameter data sets. For example, the machine learning training module may process the training data set determined at step 2208 to extract feature sets for processing through a selected machine learning algorithm for the model being trained. In some implementations, feature extraction may be executed on parameter data earlier in method 2200 for use in determining and validating the training data set selected at block 2208. At block 2212, a machine learning model may be iteratively trained to correlate parameter sets and predicted next best actions. For example, machine learning training module may apply a model training algorithm for converging on the set of coefficient values for the machine learning algorithm that results in predictions matching the corresponding labels to a desired (threshold) level of accuracy and confidence values. At step 2214, model coefficients may be determined from training. For example, the machine learning training module, based on the exit conditions for the iterative training loop being met, may select the resulting model coefficient values to determine a trained machine learning model for the selected machine learning input parameter set, algorithm, topology, and output labels. At step 2216, the trained machine learning model may optionally be validated. For example, the machine learning training module may process other labeled data through the model to confirm prediction accuracy and confidence values. At step 2218, a trained machine learning model may be deployed for production processing of new prescriptions. For example, the machine learning training module may instantiate the trained machine learning model in the corresponding component of a prescription optimization system. In some implementations, the machine learning training module may periodically return to step 2206 for retraining of each model based on retraining conditions, such as elapsed time or number of transactions, error rates, system changes, etc.

In the above description, for purposes of explanation, numerous specific details are set forth in order to provide a thorough understanding of the present disclosure. However, it should be understood that the technology described herein can be practiced without these specific details. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and operations on data within a computer memory. An algorithm is here, and generally, conceived to be a self-consistent set of operations leading to a desired result.

To facilitate description, some elements of the system and/or the methods are referred to using the labels first, second, third, etc. These labels are intended to help to distinguish the elements but do not necessarily imply any particular order or ranking unless indicated otherwise.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms including “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The technology described herein may relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, including, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of an entirely hardware implementation, an entirely software implementation, or implementations containing both hardware and software elements. For instance, the technology may be implemented in software, which includes but is not limited to firmware, resident software, microcode, etc. Furthermore, the technology can take the form of a computer program object accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

The structure, algorithms, and/or interfaces presented herein are not inherently related to any particular computer or other apparatus. Various general-purpose systems may be used with programs in accordance with the teachings herein, or it may prove convenient to construct more specialized apparatus to perform the described methods. The structure for a variety of these systems will be apparent from the description above. In addition, the techniques introduced herein are not described with reference to any particular programming language. It will be appreciated that a variety of programming languages may be used to implement the techniques as described herein.

The foregoing description has been presented for the purposes of illustration and description. It is not intended to be exhaustive or to limit the techniques to the precise form disclosed. Many modifications and variations are possible in light of the above teaching. As will be understood by those familiar with the art, the techniques may be implemented in other specific forms without departing from the spirit or essential characteristics thereof. Likewise, the particular naming and division of the modules, routines, features, attributes, methodologies and other aspects are not mandatory or significant, and the mechanisms that implement the techniques or its features may have different names, divisions and/or formats.

What is claimed is:

1. A computer-implemented method comprising:
receiving prescription information comprising a prescription for a patient;
identifying, based on the prescription information, a parameter set for determining a next best action service call from a set of next best action services;
processing the parameter set using a rules engine and a first rule set configured to conditionally determine the next best action service call in response to the parameter set meeting a next best action condition in the first rule set;
selectively processing, using a first machine learning model trained for the set of next best action services, the parameter set to determine the next best action service call; and
initiating the next best action service call.

2. The computer-implemented method of claim 1, wherein identifying the parameter set comprises determining insurance coverage parameters for the prescription and the patient.

3. The computer-implemented method of claim 1, further comprising:
selectively processing, using a second machine learning model trained for a first next best action service from the set of next best action services, the parameter set to determine a first confidence value for the first next best action service; and
determining, based on the first confidence value, whether to:
determine the next best action service call to be for the first next best action service; or
selectively process the parameter set using the first machine learning model.

4. The computer-implemented method of claim 1, further comprising:
receiving, responsive to submitting the prescription information to insurance adjudication, a rejection notification comprising a rejection code from a plurality of rejection codes; and
adding the rejection code to the parameter set.

5. The computer-implemented method of claim 4, further comprising:
selectively processing, using a plurality of specialized machine learning models, the parameter set to determine confidence values corresponding to each specialized machine learning model of the plurality of specialized machine learning models, wherein each specialized machine learning model:
is trained for a target rejection code of the plurality of rejection codes; and
generates a confidence value corresponding to a likelihood that a next best action service corresponding to that specialized machine learning model is the next best action service; and
determining, based on the confidence values, whether to:
determine the next best action service call to be for a next best action service corresponding to one of the specialized machine learning models; or
selectively process the parameter set using the first machine learning model.

6. The computer-implemented method of claim 5, wherein selectively processing the parameter set using the plurality of specialized machine learning models is executed:
responsive to processing the parameter set using the first rule set; and
prior to processing the parameter set using the first machine learning model.

7. The computer-implemented method of claim 5, wherein the plurality of specialized machine learning models comprise at least one specialized machine learning model configured to generate a change parameter for use in the next best action service call.

8. The computer-implemented method of claim 1, further comprising:
processing the parameter set using the rules engine and a second rule set configured to validate the next best action service call determined by the first machine learning model prior to initiating the next best action service call.

9. The computer-implemented method of claim 1, wherein the set of next best action services includes at least two services selected from:
therapeutic alternative recommendation service;
a quantity change service;
an insurance plan change service;
a cash pay change service;
a future fill date change service;
a prescriber request service;
a prior authorization request service; and
a customer service prompt service.

10. The computer-implemented method of claim 1, further comprising:
automatically submitting, responsive to completing the next best action service call, updated prescription information to insurance adjudication.

11. A system comprising:
at least one processor; and
a memory, the memory storing instructions which, when executed, cause the at least one processor to:
receive prescription information comprising a prescription for a patient;
identify, based on the prescription information, a parameter set for determining a next best action service call from a set of next best action services;
process the parameter set using a rules engine and a first rule set configured to conditionally determine the next best action service call in response to the parameter set meeting a next best action condition in the first rule set;
selectively process, using a first machine learning model trained for the set of next best action services, the parameter set to determine the next best action service call; and
initiate the next best action service call.

12. The system of claim 11, wherein identifying the parameter set comprises determining insurance coverage parameters for the prescription and the patient.

13. The system of claim 11, wherein the instructions further cause the at least one processor to:
selectively process, using a second machine learning model trained for a first next best action service from the set of next best action services, the parameter set to determine a first confidence value for the first next best action service; and
determine, based on the first confidence value, whether to:
determine the next best action service call to be for the first next best action service; or
selectively process the parameter set using the first machine learning model.

14. The system of claim 11, wherein the instructions further cause the at least one processor to:
receive, responsive to submitting the prescription information to insurance adjudication, a rejection notification comprising a rejection code from a plurality of rejection codes; and
add the rejection code to the parameter set.

15. The system of claim 14, wherein the instructions further cause the at least one processor to:
selectively process, using a plurality of specialized machine learning models, the parameter set to determine confidence values corresponding to each specialized machine learning model of the plurality of specialized machine learning models, wherein each specialized machine learning model:
is trained for a target rejection code of the plurality of rejection codes; and
generates a confidence value corresponding to a likelihood that a next best action service corresponding to that specialized machine learning model is the next best action service; and
determine, based on the confidence values, whether to:
determine the next best action service call to be for a next best action service corresponding to one of the specialized machine learning models; or
selectively process the parameter set using the first machine learning model.

16. The system of claim 15, wherein at least one processor is configured to execute the instructions to selectively process the parameter set using the plurality of specialized machine learning models:
responsive to processing the parameter set using the first rule set; and
prior to processing the parameter set using the first machine learning model.

17. The system of claim 15, wherein the plurality of specialized machine learning models comprise at least one specialized machine learning model configured to generate a change parameter for use in the next best action service call.

18. The system of claim 11, wherein the instructions further cause the at least one processor to:
process the parameter set using the rules engine and a second rule set configured to validate the next best action service call determined by the first machine learning model prior to initiating the next best action service call.

19. The system of claim 14, wherein the instructions further cause the at least one processor to:
automatically submit, responsive to completing the next best action service call, updated prescription information to insurance adjudication.

20. A system comprising:
at least one processor; and
a memory, the memory storing instructions which, when executed, cause the at least one processor to:
determine a set of next best action services;
determine a training data set comprised of parameter sets for a plurality of prescriptions for a plurality of patients;
label the training data set for the set of next best action services;
iteratively train a first machine learning model to correlate parameter sets to predicting a next best action service from the set of next best action services;
receive prescription information comprising a prescription for a patient;
identify, based on the prescription information, a target parameter set for determining a next best action service call from the set of next best action services;
process the parameter set using a rules engine and a first rule set configured to conditionally determine the next best action service call in response to the parameter set meeting a next best action condition in the first rule set;
selectively process, using the first machine learning model, the parameter set to determine the next best action service call; and
initiate the next best action service call.
